(19) United States
(12) Patent Application Publication
(10) Pub. No.: US 2010/0092443 A1
(43) Pub. Date: Apr. 15, 2010

(54) NOVEL COMBINATIONS OF NITROGENATED HETEROCYCLIC ANTIBACTERIAL COMPOUNDS WITH OTHER ANTIBACTERIAL COMPOUNDS AND THE USE OF SAME AS DRUGS

(75) Inventors: Premavathy Levasseur, Romainville (FR); John Lee Pace, Romainville (FR); Kenneth Coleman, Romainville (FR); John Lowther, Champigny Sur Marne (FR)

Correspondence Address: HARNESS, DICKEY & PIERCE, P.L.C., P.O. BOX 828, BLOOMFIELD HILLS, MI, 48303, US

(73) Assignee: Novexel, Romainville (FR)

(21) Appl. No.: 12/535,865

(22) Filed: Aug. 5, 2009

(30) Foreign Application Priority Data
Oct. 10, 2008 (FR) 08 05 618

(51) Int. Cl. A61K 35/74; A61K 31/551; A61K 31/70; A61K 31/43; A61K 31/65; A61K 38/14; A61K 31/397; A61K 31/545; A61P 31/04

(52) U.S. Cl. 424/93.47; 514/221; 514/23; 514/192; 514/154; 514/8; 514/210.15; 514/202; 424/93.48

(57) ABSTRACT

The invention relates to the combination of nitrogenated heterocyclic antibacterial compounds of formula (I) with other antibacterial compounds and the use of same as drugs.

The nitrogenated heterocyclic compounds are of general formula (I)

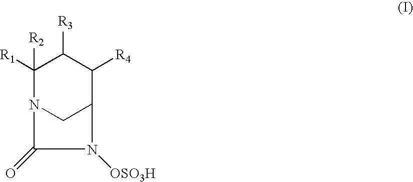

wherein R₁ represents a (CH₂)n-NH₂ or (CH₂)ₙ—NHR radical, where R is a (C₁-C₆) alkyl and n is equal to 1 or 2;
R₂ represents a hydrogen atom;
R₃ and R₄ together form an aromatic nitrogenated heterocycle with 5 apexes with 1, 2 or 3 nitrogen atoms optionally substituted by one or several R′ groups, R′ being selected in the group composed of a hydrogen atom and the alkyl radicals with 1 to 6 carbon atoms, in free form, as zwitterions, and in the form of salts of pharmaceutically acceptable inorganic or organic bases and acids.

The other antibacterial compound is selected among the group comprised of beta-lactams, monobactams or penicillins, if needed combined with a beta lactamases inhibitor, aminoglycosides, glycylcyclines, tetracyclines, quinolones, glycopeptides, lipopeptides, macrolides, ketolides, lincosamides, streptogramins, oxazolidinones, polymyxins and other compounds known to have therapeutic activity on Pseudomonas aeruginosa and Enterobacteriaceae.

### CROSS-REFERENCE TO RELATED APPLICATIONS

This application claims priority to French Patent Application No. 08 05 618, filed on Oct. 10, 2008, which is incorporated by reference herein.

### BACKGROUND AND SUMMARY

The invention concerns the combination of nitrogenated heterocyclic antibacterial compounds with other antibacterial compounds and the use of same as drugs.

The applicant discovered that novel combinations of compounds of formula (I) described and claimed in the French application 07 02663 with other antibacterial compounds have quite interesting antibacterial properties that are expressed by a synergistic effect that is as remarkable as it is unexpected. The unique character of the inventive synergistic combinations lies in particular in the fact that they exhibit excellent activity on Pseudomonas aeruginosa and Enterobacteriaceae, which are bacterial strains frequently encountered in nosocomial infections as well as in patients suffering from cystic fibrosis.

This particularly interesting and unexpected activity is not exhibited by compounds of the prior art, most notably those of the application WO 02/100860 which describes compounds comprising R₁ groups other than those nitrogenated heterocyclic compounds of formula (I) defined below. These compounds of formula (I) have been shown to be active on animal infection models, including on strains usually resistant to commonly used antibiotics. They are able to counteract the principal resistance mechanisms of bacteria, namely β-lactamases, efflux pumps and porin mutations. These compounds have the following formula:

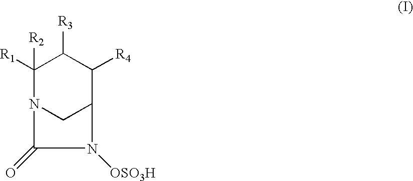

wherein R₁ represents a (CH₂)ₙ—NH₂ or (CH₂)ₙ—NHR radical, where R is a (C₁-C₆) alkyl and n is equal to 1 or 2;

R₂ represents a hydrogen atom;

R₃ and R₄ together form an aromatic nitrogenated heterocycle with 5 apexes with 1, 2 or 3 nitrogen atoms optionally substituted by one or several R′ groups, R′ being selected in the group composed of a hydrogen atom and the alkyl radicals with 1 to 6 carbon atoms;

in free form, as zwitterions, and in the form of salts of pharmaceutically acceptable inorganic or organic bases and acids.

The applicant discovered that the compounds of general formula (I) potentiate the activity of existing antibacterial compounds, in particular on Pseudomonas aeruginosa and Enterobacteriaceae. The invention thus relates to the combination of a compound of general formula (I) as defined above, in free form, as zwitterions, or in the form of salts of pharmaceutically acceptable inorganic or organic bases and acids, with another antibacterial compound. The expression “other antibacterial compound” as used herein is understood to mean notably a beta lactam, a monobactam or a penicillin, combined if needed with a beta-lactamases inhibitor, an aminoglycoside, a glycylcycline, a tetracycline, a quinolone, a glycopeptide, a lipopeptide, a macrolide, a ketolide, a lincosamide, a streptogramin, an oxazolidinone, a polymyxin and other compounds known to have therapeutic activity on Pseudomonas aeruginosa and Enterobacteriaceae.

Examples of aminoglycosides include amikacin, gentamycin and tobramycin. Examples of beta lactams include carbapenems such as imipenem, meropenem, ertapenem and the compound known as PZ-601; cephalosporins such as cefazolin, cefepime, cefotaxime, cefoxitine, ceftaroline, ceftazidime, ceftobiprole, ceftriaxone, cefuroxime and cephalexine; monobactams such as aztreonam, penicillins and combinations with inhibitors of beta-lactamases such as amoxicillin, amoxicillin/clavulanate, ampicillin, ampicillin/sulbactam, oxacillin, piperacillin, piperacillin/tazobactam, ticarcillin, ticarcillin/clavulanate and penicillin. Examples of glycylcycline and tetracycline include doxycycline, minocycline, tetracycline and tigecycline. Examples of quinolones include ciprofloxacin, gatifloxacin, grepafloxacin, levofloxacin, moxifloxacin and ofloxacin.

Examples of macrolides and ketolides include azithromycin, clarithromycin, roxythromycin and telithromycin. Examples of polymyxin include colistin and polymyxin B. Other examples of antibacterial compounds include fosfomycin, and the combination trimethoprim/sulfamethoxazole.

In the compounds of general formula (I), the expression “alkyl radical with 1 to 6 carbon atoms” as used herein is understood to mean notably the methyl, ethyl, propyl, isopropyl radical, and the linear or branched pentyl or hexyl radicals. The expression “alkenyl radical with 2 to 6 carbon atoms” as used herein is understood to mean notably the allyl radical and liner or branched butenyl, pentenyl and hexenyl radicals.

The term “aromatic heterocycle” as used herein is understood to mean notably those selected from the following list, the two bonds symbolising the junction with the nitrogenated ring (R₃R₄):

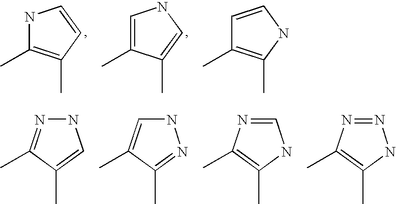

Among the acid salts of the products of formula (I), mention can be made, among other things, of those formed with inorganic acids, such as hydrochloric, hydrobromic, hydroiodic, sulphuric or phosphoric acid or with organic acids such as formic, acetic, trifluoroacetic, propionic, benzoic, maleic, fumaric, succinic, tartric, citric, oxalic, glyoxylic, aspartic, alkanesulphonic acids, such as methane and ethane sulphonic acid, arylsulphonic acids such as benzene and paratoluenesulphonic acid. Among the basic salts of the products of formula (I), mention can be made, among other things, of those formed with inorganic bases such as, for example, sodium, potassium, lithium, calcium, magnesium or ammonium hydroxide or with organic bases such as, for example, methylamine, propylamine, trimethylamine, diethylamine, triethylamine, N,N-dimethylethanolamine, tris (hydroxymethyl)amino methane, ethanolamine, pyridine, picoline, dicyclohexylamine, morpholine, benzylamine, procaine, lysine, arginine, histidine, N-methylglucamine, or phosphonium salts, such as alkyl-phosphonium, aryl-phosphoniums, alkyl-aryl-phosphonium, alkenyl-aryl-phosphonium or quaternary ammonium salts such as the tetra-n-butyl-ammonium salt.

Among the synergistic combinations as defined above, the invention notably relates to those containing compounds of formula (I) wherein R₃ and R₄ together form a pyrazolyl or triazolyl heterocycle, optionally substituted. Among these combinations, the invention notably relates to those containing compounds wherein R₁ is selected in the group composed of the groups (CH₂)ₙ—NH₂ and (CH₂)ₙ—NHCH₃, where n is as defined above, the heterocycle formed by R₃ and R₄ is substituted by a (C₁-C₆) alkyl radical. Among these combinations, the invention more particularly relates to compounds wherein R₁ represents a (CH₂)ₙ—NH₂ or (CH₂)ₙ—NHCH₃ radical, where n is as defined above and R₃ and R₄ together form a pyrazolyl ring substituted by a (C₁-C₆) alkyl radical.

Among these combinations, the invention particularly relates to those containing a compound of formula (I) selected among:
trans8-(aminomethyl)-4,8-dihydro-1-methyl-5-(sulphooxy)-4,7-methano-7H-pyrazolo[3,4-e][1,3]diazepin-6(5H)-one,
trans 8-(aminomethyl)-4,8-dihydro-5-(sulphooxy)-4,7-methano-7H-pyrazolo[3,4-e][1,3]diazepin-6(5H)-one,
trans 8-(methylaminomethyl)-4,8-dihydro-5-(sulphooxy)-4,7-methano-7H-pyrazolo[3,4-e][1,3]diazepin-6(5H)-one,
   in its free form, as a zwitterion, and salts with pharmaceutically acceptable inorganic or organic bases and acids.

Among the combinations as defined above, the invention notably relates to those containing antibacterial compounds selected among the beta-lactams or the penicillins, if needed combined with beta-lactamases inhibitors, the aminoglycosides and the polymyxins. Among these combinations, the invention notably relates to those containing antibacterial compounds selected among tobramycin, meropenem, aztreonam, cefepime, ceftazidime, piperacillin, if needed combined with tazobactam, colistin and polymyxin B.

The compounds of formula (I) can be prepared by a method comprising:
a) a step during which a compound of formula (II) is made to react with a carbonylating agent, if necessary in the presence of a base:

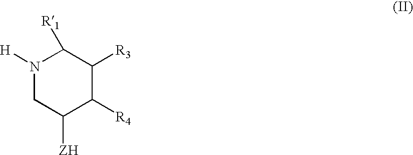

wherein:

R′₁ represents a CN, protected COOH, COOR or (CH₂)ₙR′₅ radical,

R′₅ is a protected OH, CN NH₂ or protected NHR, protected CO₂H, CO₂R radical,

n, R, R₃ and R₄ are as defined above, the aminoalkyl substituents optionally present on the heterocycle formed by R₃ and R₄ then being protected if necessary,

ZH represents a protected —NHOH group,

with the end of obtaining an intermediate compound with the formula (III):

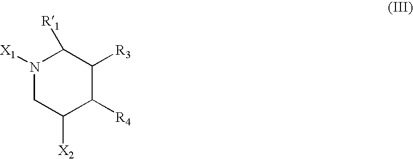

wherein:
R′₁, R₃ and R₄ have the same meanings as above and either X₁ is a hydrogen atom or a protecting group and X2 represents a —Z—CO—X₃ group, X₃ representing the rest of the carbonylating agent, or X₂ is a —ZH group and X1 represents a CO—X₃ group, X₃ being defined as above;
b) a step during which the intermediate obtained above is cyclised in the presence of a base;
   and in that:
c) if necessary, step a) is preceded and/or step b) is followed by one or several of the following reactions, in an appropriate order:

protection of the reactive functions,

deprotection of the reactive functions,

esterification

saponification,

sulphatation,

ester reduction,

alkylation,

carbamoylation,

formation of an azido group,

reduction of an azido into an amine,

salification,

ion exchange,

dividing or separating the diastereoisomers.

As a carbonylating agent, a reagent such as phosgene, diphosgene, triphosgene, an aryl chloroformiate such as phenyl or p-nitrophenyl chloroformiate, an aralkyl chloroformiate such as benzyl chloroformiate, an alkyl or alkenyl chloroformiate such as methyl or allyl chloroformiate, an alkyl dicarbonate such as tert-butyl dicarbonate, carbonyl-diimidazol and their mixtures can be used, disphosgene being preferred. The reaction preferably takes places in the presence of a base or a mixture of bases that neutralise the acid formed. The base can notably be an amine such as triethylamine, diisopropylethylamine, pyridine, dimethylaminopyridine. However, it is also possible to operate using the formula II starting product as a base. In that case an excess is used. If necessary, the formula II product is used in the form of an acid salt, for example a hydrochloride or a trifluoroacetate.

As a base in step b), it is also possible to use amines, or hydrides, alcoholates, amides or carbonates of alkaline or alkaline-earth metals. Amines can be selected for example from the list above. As a hydride, sodium or potassium hydride can notably be used. As an alkali metal alcoholate, preferably potassium t-butylate is used. As an alkali metal amide, lithium bis(trimethylsilyl)amide can notably be used. As a carbonate, sodium or potassium carbonate or bicarbonate can notably be used.

If necessary, the intermediate with the formula III can be obtained in the form of an acid salt generated during the carbonylation reaction and notably a hydrochloride. It is then used in the cyclisation reaction in this form. In preference, the cyclisation is carried out without isolating the intermediate with the formula III.

The reactions mentioned in step c) are generally conventional reactions, well known to those skilled in the art. Examples of the conditions used are described in the application WO 02/100860 and also in the application 04/052891.

The reactive functions that need protecting, if necessary, are the carboxylic acid, amine, amide, hydroxy and hydroxylamine functions. The protection of the acid function is notably provided in the form of alkyl esters, allyl, benzyl, benzhydryl or p-nitrobenzyl esters. The deprotection is carried out by saponification, acid hydrolysis, hydrogenolysis or cleavage using soluble Palladium O complexes. Examples of these protections and deprotections are supplied in the application WO 02/100860.

The protection of amines, heterocyclic nitrogens and amides is notably provided, depending on the case, in the form of benzyl or tritylated derivatives, in the form of carbamates, notably allyl, benzyl, phenyl or tertbutyl carbamates, or else in the form of silylated derivatives such as tertbutyl dimethyl, trimethyl, triphenyl or diphenyl tertbutyl-silyl derivatives, or phenylysulphonylalkyl or cyanoalkyl derivatives. The deprotection is carried out, depending on the nature of the protecting group, by sodium or lithium in liquid ammoniac, by hydrogenolysis or using soluble Palladium O complexes, by the action of an acid, or the action of tetrabutylammonium fluoride or strong bases such as sodium hydride or potassium t-butylate. The protection of hydroxylamines is carried out notably in the form of benzyl or allyl ethers. The cleaving of ethers is carried out by hydrogenolysis or using soluble Palladium O complexes.

The protection of alcohols and phenols is carried out in the conventional way, in the form of ethers, esters or carbonates. The ethers can be alkyl or alkoxyalkyl ethers, preferably methyl or methoxyethoxymethyl ethers, aryl ethers or preferably arylalkyl ethers, for example benzyl ethers, or silylated ethers, for example silylated derivatives mentioned above. The esters can be any cleavable ester known to those skilled in the art and preferably acetate, propionate, benzoate or p-nitrobenzoate. The carbonates can be for example methyl, tertbutyl, allyl, benzyl or p-nitrobenzyl carbonates.

The deprotection is carried out by means known to those skilled in the art, notably saponification, hydrogenolysis, cleavage by soluble Palladium O complexes, hydrolysis in an acid medium or, for silylated derivatives, treating with tetrabutylammonium fluoride. Examples are given in the part describing the experiments.

The sulphatation reaction is carried out by action of the SO₃-amines such as SO₃-pyridine or SO₃-dimethylformamide, working in pyridine, and the salt formed, for example the pyridine salt can then be exchanged with for example a salt of another amine, a quaternary ammonium or an alkali metal. An example is given in the part describing the experiments. The alkylation reaction is carried out by action on the hydroxylated derivatives, ester or ketone enolates, heterocyclic amines or nitrogens, depending on the case, of an alkyl sulphate or an alkyl halide or a substituted alkyl, notably by a free or esterified carboxy radical. Alkylation reactions can also be carried out by reducing amination. The salification by acids is carried out if necessary by the addition of an acid to the soluble phase of the compound. The salification by bases of the sulphooxy function can be carried out using the pyridinium salt obtained during the action of the SO₃-pyridine complex and other salts are obtained from this pyridinium salt. Ion exchange on resin can also be carried out.

The carbamoylation reaction can be carried out by using a chloroformiate or a reactive of the Boc-ON type then an amine or, if necessary, an ammoniac. An azido group can be introduced for example by the action of sodium azotide on a mesylate type intermediate or by reactions of the Mitsunobu type. The reduction of an azide group can be carried out by the action of trialkyl or triarylphosphine. The separation of enantiomers and diastereoisomers can be carried out according to techniques known to those skilled in the art, notably chromatography.

Apart from the methods described above, compounds of the formula (I) can be obtained by methods that initially use a compound of formula (II) in which R′₁, R₃, R₄ and HZ have values that lead directly (without transformation) to those of the compounds that one wishes to prepare. If necessary, the compounds of these groups that would include reactive functions such as those mentioned above are protected, and the deprotection takes place after the step b) of cyclisation or at any other appropriate moment in the synthesis. The protections and deprotections are then carried out as described above.

The compound of formula (II) is obtained by a method wherein a compound of formula (IV) is processed:

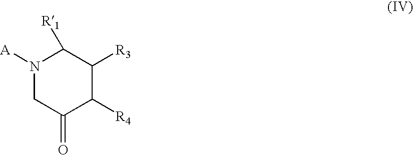

wherein R′₁, R₃ and R₄ are defined as above, and A represents a hydrogen atom or a group protecting the nitrogen, by a reducing agent, in order to obtain a compound of formula (V):

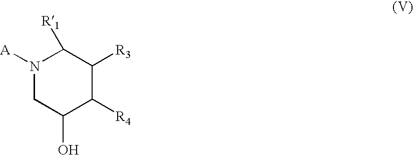

wherein A, R′₁, R₃ and R₄ keep the meanings mentioned above, wherein, if needed, the OH group is replaced by a leaving group, to obtain a compound of formula (VI):

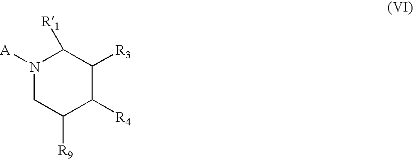

wherein A, R′₁, R₃ and R₄ keep the meanings mentioned above and R₉ represents a leaving group, that is processed with a compound of formula Z₁H₂ wherein Z₁ represents a protected —HN—OH group and then, if necessary, by a deprotection agent of the appropriate nitrogen atom.

The compound of formula (II) is further obtained by a method wherein a compound of formula (IV) is processed as defined above, by hydroxylamine protected at the hydroxyl group, to obtain a compound of formula (VII):

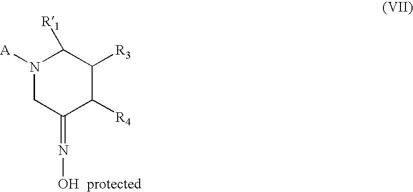

wherein A, R′₁, R′₂, R₃, R′₄, n and R′₈ are defined as above, and are made to react with a reducing agent in order to obtain a compound of formula (VIII):

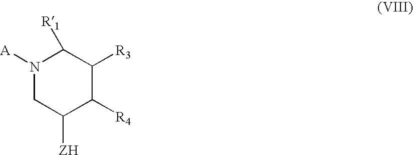

wherein A, R′₁, R₃, R₄, n″ and ZH are defined as above, that is processed, if necessary, by a deprotection agent of the appropriate nitrogen atom.

The nitrogen protection agent is notably one of those mentioned above. The reducing agent is notably an alkaline borohydride. The leaving group is notably a sulphonate, for example un mesylate or a tosylate, obtained by action of the corresponding sulphonyl chloride in the presence of a base, or a halogen, more particularly chlorine, bromine or iodine, obtained for example by action of thionyl chloride or P(C₆H₅)₃CBr₄ or PBr₃ or, in the case of an iodine atom, by the action of an alkaline iodide on a sulphonate. The deprotection agent is notably one of those mentioned above. The reducing agent used on the compound of formula (VII) is notably a sodium cyano or acetoxyborohydride.

As indicated above, the compounds of general formula (I) potentiate the activity of existing antibacterial compounds, in particular on Pseudomonas aeruginosa and Enterobacteriaceae as well as on animal infection models by strains resistant to commonly used antibacterial agents. Such a remarkable and unexpected antibiotic activity has not been observed for compounds of the prior art.

These properties make the inventive synergistic combinations suited for use as drugs, in particular in the treatment of severe infections by Pseudomonas and Enterobacteriaceae, in particular nosocomial infections and, generally, major infections in at-risk subjects. Such infections include infections of the respiratory tracts in particular, for example acute pneumonia or chronic infections of the lower respiratory tract, blood infections, for example septicaemias, acute or chronic infections of the urinary tracts, those of the auditory system, for example malignant external otitis, or chronic suppurative otitis, those of the skin and soft tissues, for example dermatitis, infected wounds, folliculitis, pyodermatitis, unresponsive acne, eye infections, for example corneal ulcer, those of the nervous system, notably meningitis and brain abscesses, cardiac infections such as endocarditis, bone and joint infections such as stenoarticular pyoarthrosis, vertebral osteomyelitis, pubic symphysitis, infections of the gastro-intestinal tube, such as necrotising enterocolitis and perirectal infections. The present invention thus further relates to the synergistic combinations as defined above as drugs and in particular as antibiotic drugs.

Among these combinations, the invention notably relates to the use as drugs of those containing compounds of formula (I) wherein R₃ and R₄ together form a pyrazolyl or triazolyl heterocycle, optionally substituted, and among these, those in which R₁ is selected in the group composed of the groups (CH₂)ₙ—NH₂ and (CH₂)ₙ—NHCH₃, where n is as defined above, the heterocycle formed by R₃ and R₄ is substituted by a (C₁-C₆) alkyl radical. Among these combinations, the invention more particularly relates to the use as drugs of those containing compounds wherein R₁ represents a (CH₂)ₙ—NH₂ or (CH₂)ₙ—NHCH₃, radical, where n is as defined above and R₃ and R₄ together form a pyrazolyl ring substituted by a (C₁-C₆) alkyl radical.

Among these combinations, the invention quite particularly relates to the use as drugs those containing at least one of the following compounds:
trans 8-(aminomethyl)-4,8-dihydro-1-methyl-5-(sulphooxy)-4,7-methano-7H-pyrazolo[3,4-e][1,3]diazepin-6(5H)-one,
trans 8-(aminomethyl)-4,8-dihydro-5-(sulphooxy)-4,7-methano-7H-pyrazolo[3,4-e][1,3]diazepin-6(5H)-one,
trans 8-(methylaminomethyl)-4,8-dihydro-5-(sulphooxy)-4,7-methano-7H-pyrazolo[3,4-e][1,3]diazepin-6(5H)-one,
   in its free form, as a zwitterion, and salts with pharmaceutically acceptable inorganic or organic bases and acids.

Among these combinations, the invention notably relates to the use as drugs of those containing antibacterial compounds selected among the aminoglycosides, the beta-lactams, the penicillins, if necessary combined with beta-lactamase inhibitors, and the polymyxins. Among these combinations, the invention notably relates to the use as drugs of those containing antibacterial compounds selected among tobramycin, meropenem, cefepime, ceftazidime, aztreonam, levofloxacin, piperacillin, if necessary combined with tazobactam, colistin and polymyxin B. The invention also relates to the pharmaceutical compositions containing as active principles a synergistic combination as defined above. These compositions can be administered orally, rectally, parenterally, in particular intramuscularly or locally by topical application on the skin and the mucosa.

The compositions according to the invention can be solid or liquid and present in pharmaceutical forms in current use in human medicine such as, for example, simple or coated tablets, capsules, granules, suppositories, injectable preparations, ointments, creams, gels; they are prepared according to the usual methods. The active principle or principles can be incorporated in the excipients usually used in these pharmaceutical compositions, such as talc, gum Arabic, lactose, starch, magnesium stearate, cocoa butter, aqueous or other media, fatty bodies of animal or plant origin, paraffin derivatives, glycols, different wetting, dispersing or emulsifying agents, preservatives. These compositions can notably take the form of a lyophilisate designed to be dissolved as required in an appropriate solvent, for example pyrogen free sterile water.

The compositions according to the invention thus include at least two active principles, which can be administered simultaneously, separately or spread over time. They can for example be provided in kit form, allowing the administration of a compound of general formula (I) and that of another antibacterial compound separately.

The dose administered of the compounds of formula (I) can vary depending on the severity and nature of the condition being treated, the particular subject, the administration route and the other antibacterial product involved. It can be, for example, between 0.250 g and 10 g per day, by oral route in humans, using the product described in example 1, or between 0.25 g and 10 g per day by intramuscular or intravenous route. The dose of the other antibacterial compound can also vary depending on the condition being treated, the particular subject, the administration route and the product involved, but generally follows the typical doses prescribed by practitioners, for example as described in the French reference Vidal. This dose can range up to 10 g per day, or even more. Nevertheless, as a result of the potentiation provided by the compounds of general formula (I) to the other antibacterial compounds, doses of the latter as part of the combination can be reduced compared to standard doses. The inventive combinations can also be used as disinfectants for surgical instruments.

### BRIEF DESCRIPTION OF DRAWINGS

FIG. 1 is a graph showing bactericidal activity of NXL 105 (compound of Example 1) alone or in combination with Ceftazidime (CAZ);

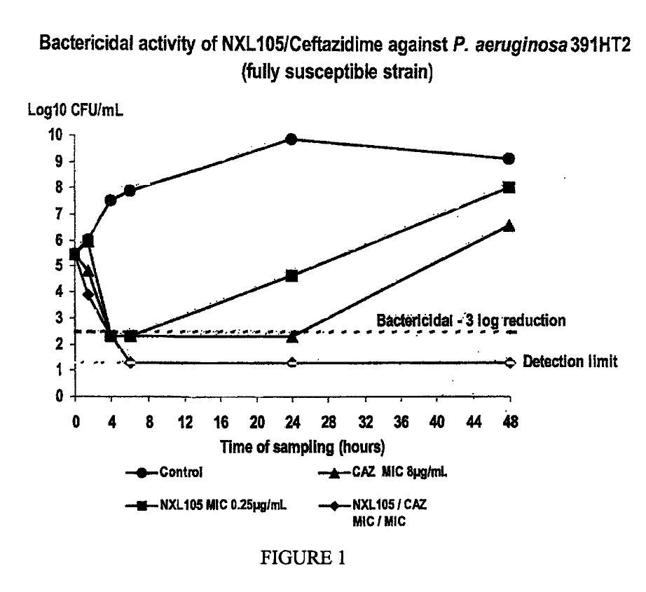

FIG. 2 is a graph showing bactericidal activity of NXL 105 (compound of Example 1) alone or in combination with Ciprofloxacine (CIPRO); and

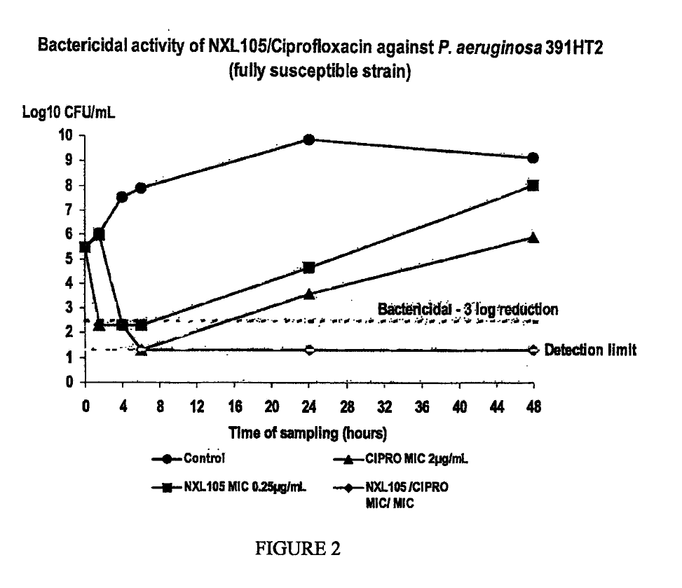

FIG. 3 is a graph showing bactericidal activity of NXL 105 (compound of Example 1) alone or in combination with Tobramycine (TOBRA).

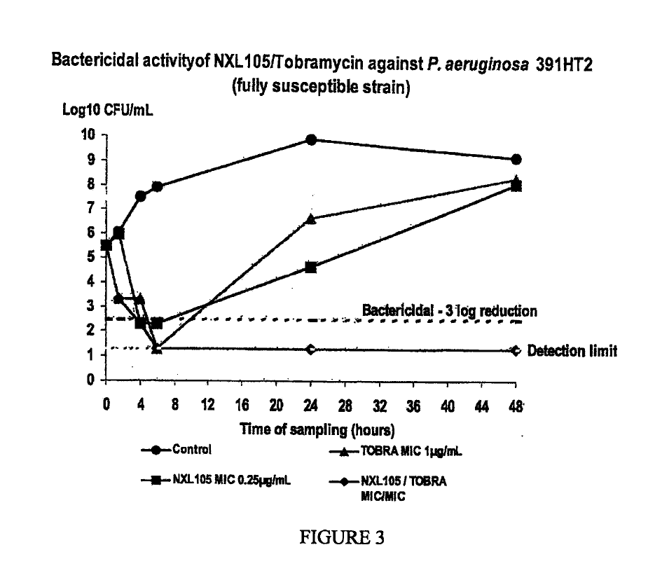

### DETAILED DESCRIPTION

The following examples illustrate the preparation of compounds of formula (I). The other antibacterial compounds are well known and available commercially.

### EXAMPLES

### Example 1

### Sodium and Trifluoroacetate Salts of trans 8-(aminomethyl)-4,8-dihydro-1-methyl-5-(sulphooxy)-4,7-methano-7H-pyrazolo[3,4-e][1,3]diazepin-6(5H)-one

### Stage A

### 4,7-dihydro-1-methyl-4-((phenylmethoxy)amino)-1H-pyrazolo[3,4-c]pyridine-6(5H),7-dicarboxylate of 6-(1,1-dimethylethyl) and 7-methyl (B)

Derivative A (4,7-dihydro-4-hydroxy-1-methyl-1H-pyrazolo[3,4-c]pyridine-6(5H),7-dicarboxylate of 6-(1,1-dimethylethyl) and 7-methyl, described in the application WO 02100860 (10 g, 32.12 mmol) is put in suspension in dichloromethane (100 ml) at ambient temperature under nitrogen and with agitation. The suspension dissolves after triethylamine is added (14.30 ml, 10.28 mmol, 3.2 eq). A solution of methane sulphonyl chloride (11.4 ml, 96.36 mmol, 3 eq) in dichloromethane (12 ml, 1 volume) is added dropwise to the reaction medium cooled to −78° C. After 30 min contact, the alcohol A is completely transformed into mesylate.

A solution of O-benzyl-hydroxylamine in dichloromethane is freshly prepared from O-benzylhydroxylamine hydrochloride (25.4 g, 160.6 mmol, 5 eq). The O-benzylhydroxylamine hydrochloride is dissolved in a mixture of O-benzylhydroxylamine hydrochloride (100 ml) and water (50 ml). A solution of 2N caustic soda (85 ml, 176.66 mmol) is added at 0° C. After 10 min of contact and decantation, the organic phase is dried on magnesium phosphate for 45 min, then concentrated to half volume. The addition of this solution to the mesylate prepared above is done at −78° C. dropwise over 1 hour. The reaction mixture is agitated allowing the temperature to increase gradually to ambient. Water (200 ml) is added and it is diluted with dichloromethane (100 ml), agitated, decanted then the aqueous phase is re-extracted with dichloromethane. The organic phase is washed with a saturated NaCl solution (200 ml), dried, then concentrated to dryness. A white amorphous powder is recovered, which after chromatography gives the B derivative expected (8.25 g, 66%).

MS (ES (+)): m/z [M⁺]=417.2

¹H NMR (400 MHz, CDCl₃): one diastereoisomer (2 rotamers) δ (ppm)=1.43 (s, 9H, tBu), 3.15 (dd, 1H, N-CH2-CH—N), 3.68/3.70 (s, 3H, CH3), 3.84 (s, 3H, CH3), 3.98 (m, 2H, N—CH2-CH—N), 4.6-4.8 (massive, 3H, NH—O—CH2-Ph and N—CH2-CH—N), 5.40/5.8 (s, 1H, CH—CO2Me), 7.22-7.31 (massive, 5H, Ph), 7.40 (s, 1H, H pyrazole)

### Stage B

### Trans 1-methyl-6-oxo-5-(phenylmethoxy)-4,5,6,8-tetrahydro-4,7-methano-1H-pyrazolo[3,4-e][1,3]diazepine-8(7H) methyl carboxylate (C)

A 4N solution of HCl/dioxane (400 ml, 15 eq) is poured into a solution of B (21 g, 50.42 mmol) dissolved in dioxane (50 ml) at ambient temperature. The reaction mixture is agitated for 30 min, then the dioxane is evaporated. The residue is taken up while being agitated in a mixture of water (100 ml) and ethyl acetate (500 ml). A solution of ammonia concentrated to 20% (42 ml) is added at 0° C. The agitation is continued for 30 min. After decantation the aqueous phase is re-extracted with ethyl acetate (2*300 ml), and the last extraction is carried out after saturation of the aqueous phase with NaCl. The organic phase is dried then concentrated. The intermediate deprotected piperidine is obtained in the form of a yellow oil (m=15.7 g, 98%) that is taken up in acetonitrile (400 ml). To this mixture cooled to 0° C., are added triethylamine (21 ml, 151.2 mmol, 3 eq), then diphosgene (3.04 ml, 25.2 mmol, 0.5 eq) dropwise over 30 min. After a night in contact at ambient temperature, the medium is concentrated then taken up with ethyl acetate (500 ml) and treated with a 10% solution of tartaric acid (200 ml). The mixture is agitated and decanted. The organic phase is washed with a solution of 10% tartaric acid (2*200 ml), then with a solution of saturated NaCl, then dried and concentrated at reduced pressure. The white product obtained (m=15.3 g, 89%) is taken up in dichloromethane (150 ml). 1-8-diazabicyclo[5.4.0]undec-7-ene (7.53 ml, 50.04 mmol) is added dropwise. The mixture is agitated for 2 hours, treated with water (200 ml), agitated, decanted. The organic phase is washed with water (2*200 ml), then with a saturated NaCl solution (1*200 ml), and dried on MgSO₄, then concentrated to dryness.

The expected derivative C is recovered (m=14.72 g, 85%), in the form of a white solid.

MS (ES (+)): m/z [M⁺]=343

¹H NMR (400 MHz, CDCl3): δ (ppm)=3.25 (d, 1H, N—CH2-CH—N), 3.45 (d, 1H, N—CH2-CH—N), 3.80 (s, 3H, CH3), 3.88 (s, 3H, CH3), 3.9 (s, 1H, N—CH2-CH—N), 4.7 (d, 1H, N—O—CH2-Ph), 5.02 (d, 1H, N—O—CH2-Ph), 5.22 (s, 1H, CH—CO2Me), 7.39-7.43 (massive, 6H, H pyrazole+Ph)

### Stage C

### 4,8-dihydro-8-(hydroxymethyl)-1-methyl-5-(phenylmethoxy)-4,7-methano-7H-pyrazolo[3,4-e][1,3]diazepin-6(5H)-one (D)

A solution of C (5 g, 14.60 mmol) in an anhydrous mixture of tetrahydrofuran (150 ml)/methanol (50 ml), under nitrogen and agitated, is cooled −10° C. Lithium borohydride (668 mg, 30.67 mmol, 1.2 eq) is added to the reaction medium. After being agitated for 2 h at −10° C., 1.2 additional eq of LiBH₄ are added. The reaction is treated cold 2 h later with a solution of 10% NaH₂PO₄. The tetrahydrofuran and the methanol are evaporated under reduced pressure (200 mbar, 40° C.). The remaining mixture is taken up with ethyl acetate (200 ml), agitated and decanted. The aqueous phase is re-extracted with 100 ml ethyl acetate. The organic phase is dried on magnesium sulphate then concentrated to dryness. The pale yellow powder obtained (6.6 g) is chromatographed on silicon dioxide (eluent-ethyl acetate) to give the derivative D (3.2 g, 10.18 mmol, 64%).

MS (ES (+)): m/z [M⁺]=315

¹H NMR (400 MHz, DMSO-_{d6}): δ (ppm)=3.16 (dd, 1H, N—CH2-CH—N), 3.48 (d, 1H, N—CH2-CH—N), 3.71 (s, 3H, CH3), 3.81-3.91 (massive, 2H, CH2OH), 4.44 (m, 1H, N—CH2-CH—N), 4.48 (m, 1H, CHCH2OH), 4.88 (m, 2H, N—O—CH2-Ph), 5.20 (m, 1H, OH), 7.35-7.40 (massive, 6H, H pyrazole+Ph).

### Stage D

### Trans 4,8-dihydro-1-methyl-8-[(methylsulfonyl)oxymethyl)]-5-(phenylmethoxy)-4,7-methano-7H-pyrazolo[3,4-e][1,3]diazepin-6(5H)-one (E)

Derivative D (2.76 g, 8.78 mmol) is dissolved in dichloromethane (100 ml) at ambient temperature under nitrogen with agitation. After cooling to 0° C., triethylamine (1.83 ml, 13.17 mmol, 1.5 eq) is added, then dropwise a solution of mesyl chloride (1.61 g, 14.05 mmol) in dichloromethane (100 ml). The ice bath is removed at the end of the addition. After one hour of contact at ambient temperature, the reaction is treated with a 10% solution of NaH₂PO₄ (80 ml) while agitating. After agitation and decantation, the aqueous phase is re-extracted with dichloromethane (50 ml). The organic phase is dried, then concentrated at reduced pressure to give the expected derivative (3.44 g, quantitative yield).

MS (ES (+)): m/z [M⁺]=393

¹H NMR (400 MHz, DMSO-d₆): δ (ppm)=3.23 (dd, 1H, N—CH2-CH—N), 3.26 (s, 3H, CH3), 3.45 (d, 1H, N—CH2-CH—N), 3.76 (s, 3H, CH3), 4.52 (m, 1H, N—CH2-CH—N), 4.58 (dd, 1H, CH—CH2-OMs), 4.66 (dd, 1H, CH—CH2-OMs), 4.88 (m, 3H, CHCH2OMs and N—O—CH2-Ph), 7.35-7.45 (massive, 6H, H pyrazole+Ph).

### Stage E

### Trans 8(azidomethyl)-4,8-dihydro-1-methyl-5-(phenylmethoxy)-4,7-methano-7H-pyrazolo[3,4-e][1,3]diazepin-6(5H)-one (F)

Sodium azide is added all at once (1.71 g, 26.3 mmol) to a solution of E (3.44 g, 8.78 mmol) in dimethylformamide (70 ml) at ambient temperature under nitrogen with agitation. The reaction medium is heated to 65° C. overnight, then treated with an aqueous solution of 10% NaH₂PO₄ (50 ml). After agitation and decantation, the aqueous phase is re-extracted with dichloromethane (2*50 ml). The organic phase is dried, then concentrated at reduced pressure to give the 3.96 g of the expected derivative F (3 g, 8.78 mmol).

MS (ES (+)): m/z [M⁺]=340

¹H NMR (400 MHz, DMSO-d₆): δ (ppm)=3.20 (dd, 1H, N—CH2-CH—N), 3.48 (d, 1H, N—CH2-CH—N), 3.66 (dd, 1H, CH—CH2-N3), 3.72 (s, 3H, CH3), 3.92 (dd, 1H, CH—CH2-N3), 4.50 (d, 1H, N—CH2-CH—N), 4.76 (dd, 1H, CHCH2-ON3), 4.89 (m, 2H, N—O—CH2-Ph), 7.35-7.45 (massive, 6H, H pyrazole+Ph).

### Stage F

### Trans [[4,5,6,8-tetrahydro-1-methyl-6-oxo-5(phenylmethoxy)-4,7-methano-7H-pyrazolo[3,4e][1,3]diazepin-8-yl]methyl]carbamate of 1,1-dimethylethyl (G)

A molar solution of trimethylphosphine (3.4 ml, 3.4 mmol) is added dropwise to a solution of F (1.15 g, 3.39 mmol) in a mixture of toluene (5 ml) and tetrahydrofuran (5 ml) at ambient temperature under nitrogen with agitation. After 3 h contact, a solution of BOC—ON (0.92 g, 3.6 mmol) in tetrahydrofuran (10 ml) is added dropwise to the reaction medium cooled to 0° C. The agitation is continued for 3 h at ambient temperature. The reaction medium is treated with a 10% aqueous solution of NaHCO₃ (50 ml). After agitation and decantation, the aqueous phase is re-extracted with ethyl acetate (50 ml). The organic phase is dried, then concentrated at reduced pressure to give 2.2 g of oil. The unrefined product is chromatographed on a silicon dioxide column (eluent cyclohexane/ethyl acetate 5/5). The expected product is obtained (0.62 g, 1.49 mmol, 70%).

MS (ES (+)): m/z [M⁺]=414

¹H NMR (400 MHz, CDCl3): δ (ppm)=1.39 (s, 9H, tBu), 3.05 (dd, 1H, N—CH2-CH—N), 3.19 (dd, 1H, CH—CH2-NHBOC), 3.27 (dd, 1H, N—CH2-CH—N), 3.72 (s, 3H, CH3), 3.78 (m, 1H, CH—CH2-NHBOC), 3.88 (d, 1H, N—CH2-CH—N), 4.48 (dd, 1H, CHCH2NHBOC), 4.79 (d, 1H, N—O—CH2-Ph), 4.92 (d, 1H, N—O—CH2-Ph), 5.18 (m, 1H, H mobile), 7.35 (s, 1H, H pyrazole), 7.37-7.48 (massive, 5H, Ph)

### Stage G

### Pyridinium Salt of trans [[4,5,6,8-tetrahydro-1-methyl-6-oxo-5-(sulphooxy)-4,7-methano-7H-pyrazolo[3,4-e][1,3]diazepin-8-yl]methyl]-carbamate of 1,1-dimethylethyl (H)

10% palladium on charcoal (140 mg) is added to a solution of G (0.6 g, 1.45 mmol) in methanol (10 ml). The reaction medium is hydrogenated for 3 h. The methanol is then evaporated at reduced pressure to give the debenzylated derivative.

MS (ES (+)): m/z [M⁺]=324

The debenzylated intermediate is taken up in pyridine (3 ml) in the presence of pyridine/sulphur trioxide complex (462 mg, 2.9 mmol). The reaction is maintained under agitation at ambient temperature overnight. The medium is then concentrated at reduced pressure. The unrefined reaction product is chromatographed on a silicon dioxide column (eluent 100% dichloromethane then gradient with methanol from 5% to 20%) to give the derivative H (0.49 g, 1.25 mmol, 84%).

MS (ES (+)): m/z [M⁻]=402

¹H NMR (400 MHz, DMSO-d₆): δ (ppm)=1.41 (s, 9H, tBu), 3.30-3.80 (massive, 4H, 2 CH2), 3.72 (s, 3H, CH3), 4.42 (dd, 1H, CHCH2ONHBOC), 4.64 (d, 1H, N—CH2-CH—N), 7.21 (m, 1H, H mobile), 7.35 (s, 1H, H pyrazole), 8.02 (dd, 2H, pyridine), 8.54 (m, 1H, pyridine), 8.91 (m, 2H, pyridine)

### Stage H

### Sodium Salt of trans [[4,5,6,8-tetrahydro-1-methyl-6-oxo-5-(sulphooxy)-4,7-methano-7H-pyrazolo[3,4-e][1,3]diazepin-8-yl]methyl]-carbamate of 1,1-dimethylethyl (I)

A suspension of 60 g of DOWEX 50WX8 resin in a solution of 2N caustic soda (300 ml) is agitated for one hour, then poured onto a chromatography column. It is eluted with demineralised water until pH neutral, then the column is conditioned with a 90/10 mixture of water/THF. Derivative H (0.49 g, 1.01 mmol) is dissolved in a minimum of water, placed on the column, then eluted with a 90/10 mixture of water/THF. The fractions containing the substrate are pooled and frozen. The frozen solution is lyophilised to lead to the expected product I (0.44 g, 1.03 mmol, 100%).

MS (ES (+)): m/z [M⁻]=402

¹H NMR (400 MHz, DMSO-d₆): δ (ppm)=1.39 (s, 9H, tBu), 3.30-3.72 (m, 7H, 2 CH2, CH3), 4.42 (m, 1H, CHCH2ONHBOC), 4.64 (s, 1H, N—CH2-CH—N), 7.16 (m, 1H, H mobile), 7.35 (s, 1H, H pyrazole).

### Stage I

### Sodium and Trifluoroacetate Salt of trans 8-(aminomethyl)-4,8-dihydro-1-methyl-5-(sulphooxy)-4,7-methano 7H-pyrazolo[3,4-e][1,3]diazepin-6(5H)-one (J)

A solution of trifluoroacetic acid (10 ml) in dichloromethane (10 ml) is poured dropwise into a solution of I (0.15 g, 0.35 mmol) in dichloromethane (5 ml) under nitrogen and cooled to 0° C. The reaction is maintained under agitation for 1 h at ambient temperature. The mixture is evaporated to dryness and taken up in a minimum of water. The solution is frozen then lyophilised to give the expected derivative J (193 mg, 0.35 mmol, 100%).

MS (ES (+)): m/z [M−]=301

¹H NMR (400 MHz, DMSO-d₆): δ (ppm)=3.32 (dd, 1H, N—CH2-CH—N), 3.33-3.37 (m, 2H, 2CH), 3.43 (d, 1H, N—CH2-CH—N), 3.74 (s, 3H, CH3), 4.73 (m, 2H, CH—CH2-NH3+), 7.41 (s, 1H, H pyrazole), 8.10 (m, 3H, NH3⁺)

### Example 2

### The Sodium and Trifluoroacetate Salt of trans 8-(amino-methyl)-4,8-dihydro-5-(sulphooxy)-4,7-methano-7H-pyrazolo[3,4-e][1,3]diazepin-6(5H)-one

### Stage A

### Trans 4,8-dihydro-8-(hydroxymethyl)-5-(phenylmethoxy)-4,7-methano-7H-pyrazolo[3,4e][1,3]diazepin-6(5H)-one

The methyl ester of trans-4,5,6,8-tetrahydro-6-oxo-5-(phenylmethoxy)-4,7-methano-7H-pyrazolo[3,4e][1,3]diazepine-8-carboxylate described in the patent WO2004/052891 (Example 1, stage K) (5 g, 15.2 mmol) is dissolved in a 1/1 mixture of anhydrous methanol/tetrahydrofuran (100 ml), under nitrogen. NaBH₄ (2.3 g, 60.9 mmol) is then added little by little. The reaction medium is agitated at ambient temperature overnight, then treated with an aqueous solution of 10% NaH₂PO₄ (100 ml). After evaporating to dryness, the reaction mixture is taken up in water. The precipitate formed is agitated overnight in ice, then filtered and dried for at least 24 h in a vacuum in presence of P₂O₅, to give the expected compound (3.3 g, 11.0 mmol, 72%) in the form of a white powder.

MS (ES(+)): m/z [M⁺]=301

¹H NMR (400 MHz, DMSO-d₆): δ (ppm)=3.18-3.50 (ABX, 2H, N—CH₂—CH—N), 3.65-3.76 (ABX, 2H, N—CH—CH₂—OH), 4.34 (t, 1H, N—CH—CH₂—OH), 4.46 (d, 1H, N—CH₂—CH—N), 4.88 (s, 2H, CH₂Ph), 7.29-7.43 (m, 5H, Ph), 7.66 (s, 1H, H pyrazole), 12.72 (broad, 1H, OH).

### Stage B

### Trans [[4,5,6,8-tetrahydro-6-oxo-5-(phenylmethoxy)-4,7-methano-7H-pyrazolo[3,4e][1,3]diazepin-8-yl]methyl]-carbamate of 1,1-dimethyl

The alcohol obtained in stage A of example 2 (1.73 g, 5.76 mmol) is dissolved in anhydrous pyridine (35 ml), under nitrogen at 0° C. Methanesulphonyl chloride (1.78 ml, 23 mmol) is added dropwise. After 2h30 of agitation at ambient temperature, the reaction medium is treated with a saturated aqueous solution of ammonium chloride (100 ml), then extracted with ethyl acetate. The combined organic phases are then washed 5 times with a saturated aqueous solution of ammonium chloride, dried on sodium sulphate, filtered then concentrated in a vacuum to give the expected dimesylated derivative in the form of a yellow oil.

The dimesylated intermediate is dissolved in anhydrous dimethylformamide (45 ml), under nitrogen, in the presence of sodium azide (1.12 g, 17.3 mmol). The reaction mixture is heated to 70° C. for 24 hours. If necessary, 1 eq of azide is added so that the conversion is complete. When the reaction is complete, the mixture is treated with a 10% aqueous solution of NaH₂PO₄ (100 ml) then extracted with dichloromethane. The combined organic phases are dried on sodium sulphate, filtered then concentrated in a vacuum to give the expected azide in the form of yellow oil.

The intermediate is put into reaction, under nitrogen, in absolute ethanol (17.5 ml). Then di-tert-butyl dicarbonate (1.38 g, 6.34 mmol), triethylsilane (1.38 ml, 8.64 mmol) and Degussa 10% palladium hydroxide on charcoal (52 mg) are added successively. After one night at ambient temperature, the reaction mixture is filtered then concentrated to give a crude yellow oil. This crude oil is purified by chromatography on a silicon dioxide column (eluent gradient CH2Cl2/MeOH 100/0 to 95/5 per 1%) to give the expected compounds (1.36 g, 3.40 mmol, 34%) as a white solid.

MS (ES(+)): m/z [M+]=401

¹H NMR (400 MHz, MeOH-d4): δ (ppm)=1.51 (s, 9H, C(CH₃)₃), 3.21-3.59 (m, 4H, N—CH₂—CH—N et N—CH—CH₂—NHBoc), 4.36 (m, 1H, N—CH—CH₂—OH), 4.46 (m, 1H, N—CH₂CH—N), 4.99 (AB, 2H, CH₂-Ph), 7.41-7.52 (m, 5H, Ph), 7.63 (s, 1H, H pyrazole).

### Stage C

### Trans [[4,5,6,8-tetrahydro-1-tert-butoxycarbamate-6-oxo-5-(phenylmethoxy)-4,7-methano-7H-pyrazolo[3,4e][1,3]diazepin-8-yl]methyl]-carbamate of 1,1-dimethyl

The compound obtained in stage B of example 2 (104 mg, 0.26 mmol) is dissolved in anhydrous dichloromethane (2.5 ml) then di-tert-butyl dicarbonate (114 mg, 0.52 mmol) and dimethylaminopyridine (32 mg, 0.26 mmol) are added to the mixture. After 1 night of agitation at ambient temperature, the reaction medium is treated with water The phases are separated then the organic phase is washed with a saturated aqueous solution of sodium chloride, dried on sodium sulphate, filtered then concentrated in a vacuum. The crude product thus obtained is purified by chromatography on silicon dioxide (eluent: CH₂Cl₂/AcOEt 90/10) to give the expected product (76 mg, 0.15 mmol, 59%).

MS (ES(+)): m/z [M+]=500

### Stage D

### Pyridinium Salt of trans [[1-tert-butoxycarbamate-4,5,6,8-tetrahydro-6-oxo-5-(sulphooxy)-4,7-methano-7H-pyrazolo[3,4e][1,3]diazepin-8-yl]methyl]-carbamate of 1,1-dimethyl

The compound obtained in stage C of example 2 (76 mg, 0.15 mmol) is dissolved, under nitrogen, in an anhydrous mixture of dimethylformamide/CH₂Cl₂ 1/3 (0.87 ml). 10% palladium on charcoal at 50% in water (49 mg) is added. After three vacuum/nitrogen purges, the reaction mixture is placed in a hydrogen atmosphere until the starting product disappears in HPLC. The mixture is then concentrated in a vacuum then co-evaporated three times with anhydrous dichloromethane and then dried in a dome in a vacuum in presence of P₂O₅ for 2 h.

The debenzylated derivative is taken up in anhydrous pyridine (0.43 ml), in nitrogen, in the presence of a pyridine/sulphur trioxide complex (48 mg, 0.30 mmol). The reaction mixture is agitated at ambient temperature until complete conversion in HPLC, then evaporated to dryness after treatment by adding water The crude product thus obtained is purified by chromatography on silicon dioxide (eluent: CH₂Cl₂/MeOH 90/10) to give the expected product (47 mg, 0.083 mmol 55%).

MS (ES(−)): m/z [M-2*BOC]=388

¹H NMR (400 MHz, MeOH-d₄): δ (ppm)=1.52 (s, 18H, 2× C(CH₃)₃), 3.50 (m, 4H, N—CH₂—CH—N et CH₂—NHBoc), 4.62 (m, 1H, CH—CH₂—NHBoc), 4.85 (d, 1H, N—CH₂—CH—N), 7.72 (s, 1H, H pyrazole).

### Stage E

### The Sodium and Trifluoroacetate Salt of trans 8-(amino-methyl)-4,8-dihydro-5-(sulphooxy)-4,7-methano-7H-pyrazolo[3,4e][1,3]diazepin-6(5H)-one

A suspension of 6 g of DOWEX 50WX8 resin in a solution of 2N caustic soda (30 ml) is agitated for 1 h, then poured onto a chromatography column. After washing with H₂O until pH neutral, the column is conditioned with a mixture of THF/H₂O 10/90. The compound obtained in stage D of example 2 (47 mg, 0.08 mmol) is dissolved in a minimum of methanol then placed on the column. After elution with a THF/H₂O 10/90 mixture, the fractions containing the expected product are pooled, frozen, then lyophilised to give the expected sodium salt.

The sodium salt is taken up in anhydrous dichloromethane (1.04 ml) in nitrogen then cooled to 0° C. A solution of trifluoroacetic acid/anhydrous dichloromethane 1/1 (2.04 ml) is added dropwise. The reaction mixture is then agitated at ambient temperature for 45 min. After evaporation to dryness then co-evaporation with anhydrous dichloromethane, the compound is taken up in water (˜2 ml) then frozen and lyophilised to give the expected salt (16 mg, 0.030 mmol, 36%) in the form of a pale yellow powder.

MS (ES(−)): m/z [M−]=288

¹H NMR (400 MHz, MeOH-d₄): δ (ppm)=3.37-3.69 (m, 4H, N—CH₂—CH—N et CH—CH₂—NH₂), 4.81 (dd, 1H, CH—CH₂—NH₂), 4.98 (d, 1H, N—CH₂—CH—N), 7.79 (s, 1H, H pyrazole).

### Example 3

### Sodium and Trifluoroacetate Salt of trans 8-(methylaminomethyl)-4,8-dihydro-1-methyl-5-(sulphooxy)-4,7-methano-7H-pyrazolo[3,4-e][1,3]diazepin-6(5H)-one

### Stage A

### Trans [[[4,5,6,8-tetrahydro-1-methyl-6-oxo-5(phenylmethoxy)-4,7-methano-7H-pyrazolo[3,4-e][1,3]diazepin-8-yl]methyl]-methylamino]trimethylphosphonium iodide

A molar solution of trimethylphosphine (1.5 ml, 1.5 mmol) is added drop by drop to a solution of the derivative obtained in stage E of example 1 (0.5 g, 1.25 mmol) in solution in tetrahydrofuran (15 ml) at ambient temperature under nitrogen and agitation. After 2 h of agitation, methane iodide (0.21 g, 3.75 mmol) is added to the reaction medium. A light yellow precipitate quickly forms. After one night of agitation at ambient temperature, the reaction medium is concentrated under reduced pressure. The crude product is triturated in dichloromethane. The precipitate is filtered to give the expected product (0.42 g, 1.04 mmol, 84%) in the form of a yellowish iodine salt.

¹H NMR (400 MHz, CDCl₃) in the form of 2 conformers: δ (ppm)=2.04 (s, 3H, CH₃P), 2.32 (s, 3H, CH₃P), 2.35 (s, 3H, CH₃P), 3.03 (s, 3H, P—NCH₃(A)-CH₂), 3.05 (s, 3H, P—NCH₃(B)-—(CH₂), 3.37 (m, 1H, N—CH₂—CH—N or CH—CH₂—N(CH₃)P), 3.44 (m, 1H, N—CH₂—CH—N or CH—CH₂—N(CH₃)P), 3.69 (m, 1H, N—CH₂—CH—N or CH—CH₂—N(CH₃)P), 3.82 (s, 3H, CH₃), 3.88 (m, 1H, N—CH₂—CH—N or CH—CH₂—N(CH₃)P), 4.05 (d, 1H, N—CH₂—CH—N), 4.59 (d, 1H, CH—CH₂—N(CH₃)P), 4.88 (d, 1H, N—O—CH₂-Ph), 5.00 (d, 1H, N—O—CH₂-Ph), 7.35 (s, 1H, H pyrazole), 7.37-7.45 (massive, 5H, Ph)

### Stage B

### Trans 8-(methylaminomethyl)-4,8-dihydro-1-methyl-5-(phenylmethoxy)-4,7-methano-7H-pyrazolo[3,4e][1,3]diazepin-6(5H)-one

To an aqueous sodium carbonate solution (2.5N, 9 ml) is added the derivative obtained in stage A of example 3 (0.42 g, 1.04 mmol). The reaction medium is agitated at 55° C. for 3 h30. After cooling at ambient temperature, the reaction medium is saturated with sodium chloride in the presence of ethyl acetate (25 ml). The aqueous phase is extracted with ethyl acetate (3×25 ml). The organic phase is dried on magnesium sulphate then concentrated under reduced pressure to yield a yellow oil (0.26 g). The crude reaction product is purified by chromatography on a silica column (eluent dichloromethane 100% then methanol gradient from 2% to 10%) to give the expected derivative (0.084 g, 0.256 mmol, 26%).

MS (ES (+)): m/z [M+H]⁺=328

¹H NMR (400 MHz, CDCl₃): δ (ppm)=2.97-3.00 (dd, 1H, N—CH₂—CH—N), 3.00 (CH—CH₂—NCH₃), 3.15 (dd, 1H, CH—CH₂—NCH₃), 3.9 (dd, 1H, N—CH₂—CH—N), 3.75 (s, 3H, CH₃), 3.98 (d, 1H, CH—CH₂—N(CH₃)Boc), 4.72 (dd, 1H, N—CH₂—CH—N), 4.90 (d, 1H, N—O—CH₂-Ph), 5.03 (d, 1H, N—O—CH₂-Ph), 7.30 (s, 1H, H pyrazole), 7.34-7.44 (massive, 5H, Ph)

### Stage C

### Trans [[4,5,6,8-tetrahydro-1-methyl-6-oxo-5-(phenylmethoxy)-4,7-methano-7H-pyrazolo[3,4-e][1,3]diazepin-8-yl]methyl]-methyl-carbamate of 1,1-dimethylethyl

The derivative obtained in stage B of example 3 (80 mg, 0.244 mmol) is put in solution in dichloromethane (1 ml) and then at ambient temperature triethyl amine (60 μL, 0.488 mmol) and di-tert-butyl dicarbonate (106 mg, 0.488 mmol) are added successively. After 4 h of agitation at ambient temperature, a solution saturated with sodium chloride (5 ml) is added to the reaction medium. The aqueous phase is extracted by dichloromethane (3×20 ml). The organic phase is dried on magnesium sulphate then concentrated under reduced pressure to give an amorphous white powder (157 mg). The crude reaction product undergoes chromatography on a silica column (eluent dichloromethane 100% then ethyl acetate gradient from 20% to 30%) to give the expected derivative (0.068 g, 0.159 mmol, 60%).

MS (ES (+)): m/z [M+H]⁺=428

¹H NMR (400 MHz, CDCl₃): δ (ppm)=1.59 (s, 9H, C(CH₃)₃), 3.05 (s, 3H, CH₃NBoc-CH₂), 3.10 (m, 3H, N—CH₂—CH—N, CH—CH₂—NBoc), 3.75 (m, 1H, N—CH₂—CH—N), 3.85 (s, 3H, CH₃), 3.99 (s, 1H, N—CH₂—CH—N), 4.75 (m, 1H, CH—CH₂—N(CH₃)Boc), 4.90 (d, 1H, N—O—CH₂-Ph), 5.02 (d, 1H, N—O—CH2-Ph), 7.37 (s, 1H, H pyrazole), 7.40-7.46 (massive, 5H, Ph)

### Stage D

### Pyridinium Salt of trans [[4,5,6,8-tetrahydro-1-methyl-6-oxo-5-(sulphooxy) 4,7-methano-7H-pyrazolo[3,4e][1,3]diazepin-8-yl]methyl]-methyl-carbamate of 1,1-dimethylethyl

While proceeding as indicated in stage G of example 1, the compound obtained in stage C of example 3 (0.068 g, 0.159 mmol) in methanol (5 ml), in the presence of 10% palladium on carbon (25 mg) leads to the debenzylated product.

MS (ES (+)): m/z [M+H]⁺=337

The debenzylated intermediate, pyridine (1 ml), pyridine/sulphur trioxide complex (50 mg, 0.318 mmol) lead to the expected salt (0.045 g, 0.090 mmol, 100%).

MS (ES (−)): m/z [M−H]⁻=416

¹H NMR (400 MHz, MeOH-d₄) in the form of 2 conformers: δ (ppm)=1.53 (s, 9H, C(CH₃)₃, 3.09 (s, 3H, CH₃(A)NHBoc), 3.10 (s, 3H, CH₃(B)NHBoc), 3.37 (m, 1H, BocN(CH₃)—CH₂—CH or N—CH₂—CH—N), 3.58 (m, 1H, BocN(CH₃)—CH₂—CH or N—CH₂—CH—N), 3.75 (s, 3H, CH₃), 3.84 (m, 1H, BocN(CH₃)—CH₂—CH or N—CH₂—CH—N), 3.90 (m, 1H, BocN(CH₃)—CH₂—CH or N—CH₂—CH—N), 4.90 (m, 2H, N—CH—CH₂—N, N—CH₂—CH—N+signal H₂O), 7.54 (s, 1H, H pyrazole), 8.16 (dd, 2H, pyridine), 8.70 (dd, 2H, pyridine), 8.94 (d, 1H, pyridine)

### Stage E

### Sodium Salt of trans[[4,5,6,8-tetrahydro-1-methyl-6-oxo-5-(sulphooxy)-4,7-methano-7H-pyrazolo[3,4-e][1,3]diazepin-8-yl]methyl]-methyl-carbamate of 1,1-dimethylethyl

While proceeding as indicated in stage H of example 1, the salt obtained in stage D of example 3 (0.045 g, 0.090 mmol), DOWEX 50WX8 resin (30 g) and 2N soda (150 ml) lead to the expected sodium salt (0.039 g, 0.090 mmol, 100%).

MS (ES (−)): m/z [M−H]⁻=416

¹H NMR (400 MHz, MeOH-d₄) in the form of 2 conformers: δ (ppm)=1.56 (s, 9H, C(CH₃)₃), 3.09 (s, 3H, CH₃(A)NHBoc), 3.10 (s, 3H, CH₃(B)NHBoc), 3.37 (m, 1H, BocN(CH₃)—CH₂—CH or N—CH₂—CH—N), 3.64 (m, 1H, BocN(CH₃)—CH₂—CH or N—CH₂—CH—N), 3.75 (s, 3H, CH₃), 3.84 (m, 1H, BocN(CH₃)—CH₂—CH or N—CH₂—CH—N), 3.93 (m, 1H, BocN(CH₃)—CH₂—CH or N—CH₂—CH—N), 4.90 (m, 2H, N—CH—CH₂—N, N—CH₂—CH—N+signal H₂O), 7.55 (s, 1H, H pyrazole).

### Stage F

### Sodium and Trifluoroacetate Salt of trans 8-(methylaminomethyl)-4,8-dihydro-1-methyl-5(sulphooxy)-4,7-methano-7H-pyrazolo[3,4e][1,3]diazepin-6(5H)-one

While proceeding as indicated in stage I of example 1, the sodium salt obtained in stage E of example 3 (0.039 g, 0.088 mmol), dichloromethane (5 ml) and a mixture of trifluoroacetic acid/anhydrous dichloromethane 1/1 (4 ml) lead to the expected product (39 mg, 0.08 mmol, 100%).

MS (ES (−)): m/z [M−H]⁻=315

¹H NMR (400 MHz, DMSO-d₆): δ (ppm)=2.76 (s, 3H, CH3NH⁺₂—CH₂), 3.30-3.50 (m, 4H, N—CH₂—CH—N, NH⁺₂—CH₂—CH), 3.75 (s, 3H, CH₃), 4.74 (m, 1H, N—CH₂—CH—N), 4.82 (d, 1H, CH—CH₂—NH⁺₂CH₃), 7.43 (s, 1H, H pyrazole), 8.67 (m, 2H, NH₃⁺)

### Example 4

### Pharmaceutical Compositions

A composition for injection was prepared containing

| | | |
|---|---|---|
| Compound of example 1: | 300 | mg |
| Tobramycin: | 500 | mg |
| Sterile aqueous excipient: | q.s.p. 5 | cm³ |

A composition for injection was prepared containing:

| | |
|---|---|
| Compound of example 1: | 200 mg |
| Ceftazidime: | 500 mg |

Determination of Bactericidal Activity

Aim

The in vitro bactericidal activity of the antibiotic is measured by showing the smallest concentration that allows the survival of 0.001% of bacteria after a single given time and over time.

Products

The products to be tested are weighed and solubilised and then the stock solution obtained is diluted in medium according to the concentrations to be tested, with each dilution having a final dilution of 1/40 (0.5 ml into a total volume of 20 ml).

Method

The minimum inhibitory concentrations (MICs) of the products to be tested (products alone and combinations) are determined in advance.
For each concentration of product to be tested as well as the control stock, an Erlenmeyer flask containing 18.5 ml Muller-Hinton medium (Ca⁺⁺) is prepared.
From an overnight culture in broth or a bacterial suspension of OD (optical density)=1, a 1/100 dilution is prepared.
The sample is cultured with agitation for 2 h at 37° C.
OD is measured: if >0.5, the sample is diluted to 1/10.
Each Erlenmeyer flask is inoculated with 1 ml of the agitated culture or its dilution. The initial inoculum should be 1×10⁶ CFU/ml.
The various antibiotic solutions are added at a volume of 0.5 ml and 0.5 ml of medium in the control Erlenmeyer flask.
With a volume of 0.1 ml, the control Erlenmeyer flask is numbered=TO.
The flask is incubated at 37° C. with agitation.
At each sampling point (2, 4, 6, 24, 48 hours), a volume of 0.1 ml is sampled from each Erlenmeyer flask and numbered.
Plates for all the numbered samples are incubated (24 h-48 h) at 37° C.

Parameters Measured

The colonies are counted.

Curves are plotted for CFU/ml as a function of time.

Bactericidal effect=3 log decrease compared to the initial inoculum.

### BIBLIOGRAPHY

PETERSON L. R., SHANHOLTZER C. J.
Tests for bactericidal effects of antimicrobial agents: technical performance and clinical relevance.
Clin. Microb. Rev., 1992, 5, 420-432
COURVALIN P., DRUGEON H., FLANDROIS J. P., GOLDSTEIN F.
Bactéricidie. Aspects théoriques et thérapeutiques.
Ed. Maloine, Paris, 1991.

Bactericidal activity was evaluated on a sensitive strain of P. aeruginosa (391 HT2)

MICs are determined on microplates:

| | |
|---|---|
| Ceftazidime/CAZ: | 2 μg/ml |
| Ciprofloxacine/CIPRO: | 1 μg/ml |
| Tobramycin/TOBRA: | 1 μg/ml |
| Product of example 1 (NXL105): | 0.25 μg/ml |

For the bactericidal tests, MICs are determined in a volume of 10 ml-bactericidal conditions (exponential bacterial growth):

| | |
|---|---|
| CAZ: | 8 μg/ml |
| CIPRO: | 2 μg/ml |
| TOBRA: | 1 μg/ml |
| Product of example 1: | 0.25 μg/ml |

The bactericidal activities presented on plates 1 to 3 in the annex are evaluated after 48H, either for the product of example 1 alone, or for a combination. They show a total absence of bacterial re-growth after 48 h for the combinations.

Demonstration of Synergistic Activity—Determination of MICs:

In vitro activity, method of dilutions in liquid medium:

A series of test 96-well microtiter plates are prepared in which the same quantity of sterile nutritive medium is distributed. Increasing quantities of the compound to be studied, namely the antibacterial compound alone and the inventive combination with the compound of formula (I) of example 1, are distributed in each plate in the respective proportions 2:1 and 4:1, and then each plate is inoculated with a bacterial strain of Pseudomonas aeruginosa. After 24 h of incubation in a 37° C. oven, growth inhibition is evaluated by transillumination, which makes it possible to determine the minimum inhibitory concentrations (MICS) expressed in μg/ml.

In all of the tests below (MIC and FIC):

Ceftazidime=CAZ

Meropenem=MRP

Aztreonam=AZT

Levofloxacin=LVX

Compound of example 1=NXL105

| | | | Meropenem | | | Aztreonam | | | Ceftazidime | | |
|---|---|---|---|---|---|---|---|---|---|---|---|
| | | NXL105 | | +NXL105 | | | +NXL105 | | | +NXL105 | |
| Species | Alias | Alone | Alone | 2:1 | 4:1 | Alone | 2:1 | 4:1 | Alone | 2:1 | 4:1 |
| P. aeruginosa | ATCC 27853 | 0.060 | 1.000 | 0.250 | 0.500 | 8 | 0.25 | 0.5 | 0.5 | 0.5 | 0.5 |
| P. aeruginosa | 391KB135 | 1.000 | >32 | 4.000 | 16.000 | >32 | 2 | 2 | >32 | 2 | 8 |
| P. aeruginosa | 391KB136 | 0.500 | >32 | 1.000 | 4.000 | >32 | 1 | 2 | >32 | 1 | 2 |
| P. aeruginosa | 391KB137 | 0.500 | >32 | 2.000 | 2.000 | 32 | 1 | 2 | >32 | 0.5 | 2 |
| P. aeruginosa | 391KB138 | 0.500 | >32 | 1.000 | 2.000 | 32 | 2 | 4 | >32 | 0.5 | 2 |
| P. aeruginosa | 391KB139 | 2.000 | >32 | 1.000 | 4.000 | >32 | 2 | 4 | >32 | 2 | 4 |
| P. aeruginosa | 391KB140 | 2.000 | 16.000 | 0.500 | 1.000 | 8 | 0.5 | 1 | 16 | 0.5 | 2 |
| P. aeruginosa | 391KB141 | 4.000 | >32 | 1.000 | 2.000 | 16 | 1 | 2 | >32 | 1 | 2 |
| P. aeruginosa | 391KB142 | 1.000 | >32 | 2.000 | 4.000 | >32 | 2 | 4 | >32 | 2 | 4 |
| P. aeruginosa | 391KB143 | 0.500 | >32 | 1.000 | 2.000 | >32 | 1 | 2 | >32 | 2 | 4 |
| P. aeruginosa | 391KB144 | 16.000 | >32 | 1.000 | 2.000 | 32 | 1 | 4 | >32 | 2 | 4 |
| P. aeruginosa | 391QBR2 | >32 | >32 | 1.000 | 4.000 | >32 | >32 | >32 | >32 | 16 | 16 |
| P. aeruginosa | 391QBR3 | 2.000 | 32.000 | 1.000 | 2.000 | >32 | 2 | 4 | >32 | 1 | 4 |
| P. aeruginosa | 391QBR4 | 2.000 | 16.000 | 2.000 | 8.000 | >32 | >32 | >32 | >32 | 16 | 32 |
| P. aeruginosa | 391QBR5 | 1.000 | 32.000 | 2.000 | 2.000 | >32 | 2 | 4 | >32 | 4 | 32 |
| P. aeruginosa | 391QBR7 | 2.000 | 32.000 | 2.000 | 4.000 | >32 | 1 | 2 | >32 | 1 | 4 |
| P. aeruginosa | 391QBR8 | 2.000 | 16.000 | 1.000 | 2.000 | >32 | 2 | 4 | >32 | 2 | 4 |
| P. aeruginosa | 391QBR9 | 2.000 | 16.000 | 2.000 | 4.000 | >32 | 4 | 8 | >32 | 2 | 4 |
| P. aeruginosa | 391QBR10 | 4.000 | 4.000 | 0.500 | 0.500 | >32 | 0.5 | 1 | >32 | 0.5 | 1 |
| P. aeruginosa | 391KB62 | 4.000 | >32 | 4.000 | 8.000 | >32 | 8 | 16 | >32 | 4 | 16 |
| P. aeruginosa | 391KB21 | 2.000 | 8.000 | 4.000 | 4.000 | >32 | 2 | 4 | >32 | 2 | 8 |
| P. aeruginosa | 391KB114 | 4.000 | 16.000 | 4.000 | 8.000 | >32 | 8 | 16 | >32 | 4 | 16 |
| P. aeruginosa | 391KB91 | 0.500 | 16.000 | 1.000 | 2.000 | >32 | 1 | 2 | >32 | 1 | 2 |
| P. aeruginosa | 391KB99 | 0.500 | 16.000 | 1.000 | 1.000 | >32 | 1 | 1 | >32 | 1 | 2 |
| P. aeruginosa | 391KB9 | 0.250 | 4.000 | 0.500 | 1.000 | >32 | 1 | 1 | >32 | 1 | 1 |
| P. aeruginosa | 391KB90 | 8.000 | 16.000 | 2.000 | 2.000 | >32 | 2 | 2 | >32 | 4 | 4 |
| P. aeruginosa | 391KB38 | 1.000 | 8.000 | 1.000 | 1.000 | >32 | 1 | 2 | >32 | 2 | 4 |
| P. aeruginosa | 391KB68 | 0.500 | 1.000 | 0.250 | 0.500 | 32 | 1 | 1 | >32 | 0.5 | 2 |
| P. aeruginosa | 391KB105 | 1.000 | 4.000 | 0.500 | 1.000 | >32 | 1 | 1 | >32 | 1 | 2 |
| P. aeruginosa | 391KB127 | 0.500 | 2.000 | 0.500 | 1.000 | >32 | 1 | 2 | >32 | 1 | 2 |
| P. aeruginosa | 391KB33 | 0.500 | 4.000 | 0.500 | 1.000 | >32 | 2 | 2 | 32 | 1 | 2 |
| P. aeruginosa | 391KB14 | 1.000 | 8.000 | 0.500 | 1.000 | >32 | 1 | 2 | 32 | 1 | 2 |
| P. aeruginosa | 391KB107 | 1.000 | 32.000 | 2.000 | 2.000 | >32 | 1 | 4 | >32 | 1 | 2 |
| P. aeruginosa | 391KB103 | 0.500 | 8.000 | 0.500 | 1.000 | >32 | 1 | 2 | 32 | 1 | 2 |
| P. aeruginosa | 391KB106 | 2.000 | 4.000 | 0.500 | 0.500 | >32 | 2 | 4 | 32 | 2 | 4 |
| P. aeruginosa | 391K767 | 1.000 | 2.000 | 0.500 | 0.500 | 8 | 0.5 | 1 | 2 | 0.5 | 1 |
| P. aeruginosa | 391K1523 | 2.000 | 2.000 | 0.250 | 0.250 | 8 | 0.25 | 0.5 | 16 | 0.25 | 0.5 |
| P. aeruginosa | 391K1455 | 2.000 | 4.000 | 0.500 | 0.500 | 32 | 0.5 | 2 | 32 | 0.5 | 1 |
| P. aeruginosa | 391K1536 | 2.000 | 2.000 | 0.500 | 0.500 | 32 | 0.25 | 1 | 32 | 0.5 | 0.5 |
| P. aeruginosa | 391K1525 | 4.000 | 2.000 | 0.500 | 0.500 | 32 | 0.5 | 2 | 32 | 0.5 | 1 |
| P. aeruginosa | 391K2415 | 2.000 | 2.000 | 0.250 | 0.500 | 32 | 0.5 | 2 | 32 | 0.5 | 1 |
| P. aeruginosa | 391K2376 | 2.000 | 2.000 | 0.500 | 0.500 | 32 | 0.5 | 2 | 32 | 0.5 | 1 |
| P. aeruginosa | 391K2379 | 4.000 | 4.000 | 0.500 | 0.500 | 16 | 0.5 | 1 | 32 | 0.5 | 1 |
| P. aeruginosa | 391HG38 | 32.000 | 32.000 | 8.000 | 8.000 | >32 | 8 | 8 | >32 | 16 | 16 |
| P. aeruginosa | 391HG39 | 4.000 | 0.060 | 0.060 | 0.060 | 0.5 | 0.5 | 0.25 | 1 | 0.5 | 1 |
| P. aeruginosa | 391HG58 | 1.000 | 32.000 | 2.000 | 4.000 | >32 | 2 | 4 | >32 | 2 | 4 |
| P. aeruginosa | 391HG123 | >32 | 32.000 | 4.000 | 4.000 | >32 | 4 | 4 | 32 | 1 | 4 |
| P. aeruginosa | 391HG158 | 32.000 | 32.000 | 4.000 | 16.000 | >32 | 8 | 16 | >32 | 8 | 16 |
| P. aeruginosa | 391HG271 | 32.000 | >32 | 4.000 | 8.000 | >32 | 2 | 4 | >32 | 2 | 4 |
| P. aeruginosa | C3719 | >32 | >32 | 16.000 | 16.000 | >32 | 16 | 16 | >32 | 16 | 32 |
| P. aeruginosa | 391HG118 | 0.125 | 8.000 | 0.500 | 1.000 | 16 | 0.5 | 0.5 | 4 | 0.25 | 0.5 |
| P. aeruginosa | 391HG2 | 1.000 | >32 | 4.000 | 8.000 | >32 | 4 | 4 | >32 | 2 | 4 |
| P. aeruginosa | 391HG21 | 0.500 | >32 | 1.000 | 2.000 | 32 | 1 | 2 | >32 | 1 | 2 |
| P. aeruginosa | PA2192 | 0.125 | 8.000 | 0.060 | 0.060 | 0.5 | 0.25 | 0.25 | 2 | 0.125 | 0.25 |
| P. aeruginosa | 391HG329 | 2.000 | >32 | 2.000 | 2.000 | 32 | 1 | 2 | >32 | 1 | 4 |

| | | | NXL- | Ceftazidime | | | Aztreonam | | | Meropenem | | |
|---|---|---|---|---|---|---|---|---|---|---|---|---|
| | | Mechanism of | 105 | | +NXL105 | | | +NXL105 | | | +NXL105 | |
| Species | Alias | Resistance | Alone | Alone | 2:1 | 4:1 | Alone | 2:1 | 4:1 | Alone | 2:1 | 4:1 |
| E. coli | 2138 | KPC-2 + TEM-1 | >32 | >32 | 0.25 | 0.25 | 32 | 0.25 | 0.5 | 8 | 0.125 | 0.125 |
| K. pneumoniae | YC | KPC-2 | >32 | >32 | 1 | 2 | >32 | 0.5 | 1 | 32 | 0.5 | 2 |
| E. cloacae | 7506 | KPC-2 + TEM-1 + | 32 | >32 | 1 | 1 | >32 | 1 | 1 | 16 | 1 | 2 |
| | | KLUC-2 | | | | | | | | | | |
| K. pneumoniae | VAKP | KPC-2 | >32 | >32 | 0.125 | 1 | 16 | 2 | 1 | 32 | 8 | 2 |
| K. pneumoniae | CL5761 | KPC-3 | >32 | >32 | 2 | 4 | >32 | 4 | 8 | >32 | 2 | 4 |
| K. pneumoniae | CL5762 | KPC-3 | >32 | >32 | 1 | 2 | >32 | 0.5 | 1 | >32 | 1 | 2 |
| K. pneumoniae | CL5763 | KPC-3 | >32 | >32 | 4 | 8 | >32 | 4 | 4 | >32 | 4 | 8 |
| K. pneumoniae | VA8 | KPC-3 | >32 | >32 | 0.06 | 0.25 | 32 | <=0.03 | 0.06 | <=0.03 | <=0.03 | <=0.03 |
| K. pneumoniae | ATCC | SHV-18 | >32 | >32 | 0.25 | 0.5 | 8 | 0.5 | 1 | 0.06 | <=0.06 | <=0.03 |
| | 700603 | | | | | | | | | | | |
| K. pneumoniae | Patient | VIM-4 + CTX-M-15 = | 16 | >32 | <=0.03 | 0.25 | 16 | 0.06 | 0.125 | 16 | 8 | 4 |
| | 1BHR | CMY-4 + TEM-1 | | | | | | | | | | |
| K. pneumoniae | Patient F | VIM-1 + SHV-5 | >32 | >32 | >32 | >32 | >32 | 1 | 2 | >32 | 32 | >32 |
| K. pneumoniae | 9701 | CMY-4 + TEM-1 | >32 | >32 | 2 | 2 | 16 | 1 | 2 | 0.125 | 0.06 | 0.06 |
| K. pneumoniae | SLK54 | ACC-1 + TEM-1 | >32 | >32 | 1 | 1 | 2 | <=0.03 | 0.125 | 0.06 | 0.06 | 0.06 |
| K. pneumoniae | 1734 | FOX-3 + TEM-1 | >32 | >32 | 0.06 | 0.06 | 8 | <=0.03 | 0.125 | 0.125 | <=0.03 | <=0.03 |
| K. pneumoniae | TN58467 | DHA-1 + SHV-2a + | >32 | >32 | 0.5 | 1 | 16 | <=0.03 | 0.06 | 0.25 | 0.06 | 0.06 |
| | | TEM-1 | | | | | | | | | | |
| K. pneumoniae | KOL | MOX-2 + SHV-5 + | >32 | >32 | <=0.03 | <=0.03 | 32 | <=0.03 | 0.125 | 0.06 | <=0.03 | <=0.03 |
| | | TEM-1 | | | | | | | | | | |
| K. pneumoniae | Tunisie | CTX-M-15 + TEM-1 + | 16 | >32 | 0.5 | 1 | >32 | 0.25 | 0.25 | 0.125 | 0.06 | 0.06 |
| | clone K4 | OXA-1 | | | | | | | | | | |
| K. pneumoniae | Tunisie | CTX-M-16 + OXA-1 | >32 | >32 | 1 | 1 | >32 | 2 | 2 | 0.125 | <=0.03 | <=0.03 |
| | clone K1 | | | | | | | | | | | |
| K. pneumoniae | KP04 | CTX-M-14 | >32 | 32 | 0.5 | 1 | 16 | 1 | 1 | 0.125 | 0.06 | 0.06 |
| K. pneumoniae | 157 | SHV-5 + TEM-26 | >32 | >32 | 0.06 | 0.125 | 16 | <=0.03 | 0.125 | <=0.03 | <=0.03 | <=0.03 |
| K. pneumoniae | 449 | SHV-1 + TEM-2 + PER | 32 | >32 | 0.125 | 0.25 | 32 | <=0.03 | 0.25 | <=0.03 | <=0.03 | <=0.03 |
| K. pneumoniae | 444 | SHV-5 + TEM-2 + PER | >32 | >32 | <=0.03 | 0.06 | 8 | 0.5 | 0.25 | 0.125 | <=0.03 | <=0.03 |
| K. pneumoniae | 441 | SHV-1 + TEM-2 + PER | >32 | >32 | 0.125 | 0.5 | 16 | 0.125 | 0.06 | 0.06 | <=0.03 | <=0.03 |
| K. pneumoniae | 60 | SHV-2 + TEM-2 + PER | 32 | >32 | 0.06 | 0.06 | 16 | 0.125 | 0.125 | 0.06 | <=0.03 | <=0.03 |
| K. pneumoniae | 427 | SHV-1 + TEM-1B + | >32 | >32 | <=0.03 | 0.25 | 4 | 0.06 | 0.06 | 0.06 | <=0.03 | <=0.03 |
| | | CTX-M-3 | | | | | | | | | | |
| K. pneumoniae | 465 | TEM-1B + CTX-M-2 | >32 | >32 | 0.5 | 0.5 | 32 | 0.5 | 0.5 | 0.25 | 0.06 | 0.06 |
| K. pneumoniae | 253 | SHV-2 + TEM-12 + | >32 | >32 | 0.125 | 0.25 | 16 | 0.25 | 1 | 0.06 | <=0.03 | 0.06 |
| | | CTX-M-2 | | | | | | | | | | |
| K. pneumoniae | 181 | SHV-5 + TEM-10 | >32 | >32 | 0.5 | 0.5 | 32 | 0.25 | 0.5 | 4 | 0.25 | 0.5 |
| K. pneumoniae | 243 | SHV-5 + TEM-63 | >32 | >32 | 0.25 | 0.5 | 32 | 0.125 | 0.25 | 0.125 | 0.06 | 0.06 |
| K. pneumoniae | 238 | SHV-2 + TEM-12 + | >32 | >32 | 0.25 | 0.25 | 32 | 1 | 1 | <=0.03 | <=0.03 | <=0.03 |
| | | CTX-M-2 | | | | | | | | | | |
| K. pneumoniae | 236 | SHV-5 + TEM-10 | >32 | >32 | <=0.03 | 0.125 | 16 | 0.125 | 0.125 | 0.125 | <=0.03 | <=0.03 |
| K. pneumoniae | 26 | SHV-5 | 32 | >32 | 0.125 | 0.25 | 16 | <=0.03 | 1 | <=0.03 | <=0.03 | <=0.03 |
| K. oxytoca | 16944 | OXY-2, TEM-1 | >32 | >32 | 0.5 | 0.5 | 32 | 0.5 | 0.5 | 0.06 | <=0.03 | <=0.03 |
| K. oxytoca | 1431 | TEM-129 | 32 | >32 | 0.25 | 0.25 | >32 | 0.5 | 0.5 | 0.06 | 0.06 | 0.06 |
| E. cloacae | | AmpC | >32 | 32 | 8 | 16 | 16 | 4 | 8 | 0.125 | 0.06 | 0.125 |
| E. cloacae | | AmpC | 32 | >32 | 0.25 | 0.5 | 16 | 0.125 | 0.125 | 0.5 | 0.125 | 0.25 |
| E. coli | | CTX-M-15 + TEM-1 + | >32 | >32 | <=0.03 | <=0.03 | 4 | <=0.03 | <=0.03 | <=0.03 | <=0.03 | <=0.03 |
| | | OXA-1 | | | | | | | | | | |
| E. coli | IND | VEB-1 + CMY-2 | 16 | >32 | 0.125 | 2 | >32 | 0.25 | 0.25 | 0.06 | 0.06 | 0.06 |
| E. coli | TN13 | CTX-M-14 + CMY-2 + | 32 | >32 | 4 | 2 | 16 | <=0.03 | 0.06 | 0.25 | <=0.03 | 0.06 |
| | | TEM-1 | | | | | | | | | | |
| E. coli | Tunisie | CTX-M-16 + TEM-1 | >32 | >32 | 2 | 4 | 32 | 2 | 2 | 0.125 | <=0.03 | <=0.03 |
| | clone E4 | | | | | | | | | | | |
| E. coli | | Cephalosporinase | >32 | >32 | 2 | 8 | 16 | <=0.03 | 0.06 | 0.06 | <=0.03 | <=0.03 |
| E. coli | TN06 | CTX-M-2 + TEM-1 | 32 | 16 | 0.25 | 0.25 | 8 | 0.5 | 0.5 | 0.06 | <=0.03 | <=0.03 |
| E. coli | | TEM-3 | >32 | 32 | 0.06 | 0.06 | 8 | <=0.03 | 0.06 | <=0.03 | <=0.03 | <=0.03 |
| E. coli | | SHV-4 | >32 | >32 | 0.06 | 0.125 | 16 | 0.06 | <=0.03 | <=0.03 | <=0.03 | <=0.03 |
| C. freundii | | AmpC | 16 | >32 | <=0.03 | 0.125 | 8 | 0.06 | 0.125 | 0.25 | <=0.03 | <=0.03 |
| C. freundii | | AmpC | 32 | >32 | <=0.03 | 1 | 32 | 0.125 | 0.5 | 0.06 | <=0.03 | <=0.03 |
| C. freundii | | AmpC | >32 | >32 | 2 | 8 | 16 | 4 | 8 | 0.125 | 0.06 | <=0.03 |
| C. freundii | | AmpC + TEM-1 | 16 | >32 | 1 | 2 | 16 | 0.5 | 0.5 | 0.06 | <=0.03 | <=0.03 |
| C. freundii | | AmpC | 32 | 16 | <=0.03 | <=0.03 | 4 | <=0.03 | <=0.03 | 0.06 | <=0.03 | <=0.03 |
| E. aerogenes | EAR2 | Case-R + FEP-R | >32 | >32 | >32 | >32 | 16 | 4 | 4 | 0.125 | 0.125 | 0.125 |

Demonstration of Synergistic Activity—Determination of Fractional Inhibitory Concentrations (FICs)

Checkerboard Technique for Determination of Antibiotic Synergy

Objective: The purpose of the study was to determine the concentration of a compound A, required to reduce the MIC of a compound B by one-half, one-quarter, one-eighth, one-sixteenth, and one-thirty-second against strains of Enterobacteriaceae and non-Enterobacteriaceae species resistant to compound B.

The above objective was accomplished by the checkerboard technique. This technique is used to assess antimicrobial combinations.
This technique consists of titrating the compound A, an inhibitor, in a serial dilution across a microtiter plate, while at the same time titrating compound B in a serial dilution down the microtiter plate. The plate is then inoculated with the bacterial strain in question and allowing the bacteria to grow up overnight. Each well in this microtiter checkerboard contains a different combination of concentrations of the inhibitor and the antibacterial compound allowing a full determination of any synergy between the two.

Reading of Plates

Plates were scored for growth in each well. End points (MICs) where there was no growth in each row were determined and the concentrations of compound A and compound B at each of these growth-negative wells were then used to determine levels of synergy.
Synergy is represented as FIC indicies which is the Fractional Inhibitory Concentration of the combinational drugs.

Calculations of the Fractional Inhibitory Concentration (FIC) Index for Combinations of Two Antimicrobial Agents

(A)/(MIC_{A})+(B)/(MIC_{B})=FIC_{A}+FIC_{B}=FIC index

(A) is the concentration of compound A in a well that is the lowest concentration of antibiotic A inhibiting growth in the row when in an assay well also containing compound B. (MIC_{A}) is the lowest concentration of compound A alone that inhibits growth. FIC_{A} is the fractional inhibitory concentration of drug A.
(B), (MIC_{B}), and FIC_{B} are defined in the same fashion for compound B. If the FIC index value is <=0.5 it is considered as synergy.

Enterobacteriaceae

| | | | FIC Index at xMIC | | | | | |
|---|---|---|---|---|---|---|---|---|
| Species | Mechanism of Resistance | Combination | 0.5x | 0.25x | 0.125x | 0.06x | 0.03x | 0.015x |
| E. coli Tunisia E4 | CTX-M-16 + TEM-1 | CAZ + NXL105 | 0.5004 | 0.25* | 0.125* | 0.06* | 0.03* | 0.015* |
| | | MRP + NXL105 | 0.51 | 0.375* | 0.26* | 0.19* | | |
| | | AZT + NXL105 | 0.5004 | 0.25* | 0.125* | 0.07* | 0.039* | 0.031* |
| E. coli TN06 | CTX-M-2 + TEM-1 | CAZ + NXL105 | 0.5009 | 0.25* | 0.125* | 0.06* | 0.03* | 0.0175* |
| | | MRP + NXL105 | 0.5009 | 0.375* | 0.26* | 0.566 | | |
| | | AZT + NXL105 | 0.5004 | 0.25* | 0.125* | 0.06* | 0.03* | 0.016* |
| K. pneumoniae 465 | CTX-M-2 + TEM-1B | CAZ + NXL105 | 0.5009 | 0.25* | 0.125* | 0.064* | 0.032* | 0.016* |
| | | MRP + NXL105 | 0.5009 | 0.24* | 0.62 | 0.564 | | |
| | | AZT + NXL105 | 0.5009 | 0.25* | 0.125* | 0.06* | 0.032* | 0.0175* |
| E. cloacae 293HT96 | AmpC | CAZ + NXL105 | 0.5009 | 0.25* | 0.125* | 0.064* | 0.032* | 0.016* |
| | | MRP + NXL105 | 0.5009 | 0.25* | 0.37* | 0.564 | | |
| | | AZT + NXL105 | 0.5009 | 0.25* | 0.125* | 0.06* | 0.03* | 0.016* |
| E. cloacae 293GR38 | AmpC | CAZ + NXL105 | 0.5009 | 0.257* | 0.156* | 0.31* | 0.53 | 0.51 |
| | | MRP + NXL105 | 0.75 | 0.43* | 0.62 | 0.56 | 0.53 | |
| | | AZT + NXL105 | 0.507 | 0.31* | 0.375* | 0.56 | 0.53 | 0.51 |
| E. coli 250SUJ1 | KPC-2 + TEM-1 | CAZ + NXL105 | 0.5005 | 0.25* | 0.1255* | 0.061* | 0.032* | 0.02* |
| | | MRP + NXL105 | 0.5005 | 0.2505* | 0.1255* | 0.061* | 1.03 | |
| | | AZT + NXL105 | 0.5009 | 0.25* | 0.125* | 0.06* | 0.03* | 0.0175* |
| K. pneumoniae 283KB7 | KPC-2 | CAZ + NXL105 | 0.5005 | 0.254* | 0.129* | 0.064* | 0.038* | 0.023* |
| | | MRP + NXL105 | 0.05005 | 0.2505* | 0.127* | 0.062* | 0.034* | 0.019* |
| | | AZT + NXL105 | 0.504 | 0.25* | 0.125* | 0.066* | 0.033* | 0.0166* |

Pseudomonas

| | | | FIC Index at xMIC | | | | | |
|---|---|---|---|---|---|---|---|---|
| Species | Mechanism of Resistance | Combination | 0.5x | 0.25x | 0.125x | 0.06x | 0.03x | 0.015x |
| P. aeruginosa 391QBR2 | ampC | CAZ + NXL105 | 0.5004 | 0.252* | 0.129* | 0.068* | 0.045* | 0.03* |
| | | MRP + NXL105 | 0.508 | 0.265* | 0.156* | 0.185* | 0.28* | 0.265* |
| | | LVX + NXL105 | 0.508 | 0.281* | 0.375* | 1.06 | | |
| P. aeruginosa 391QBR6 | ampC | CAZ + NXL105 | 0.5625 | 0.3125* | 0.25* | 0.31* | 0.28* | 0.265* |
| | | MRP + NXL105 | 0.53 | 0.5* | 0.375* | | | |
| | | LVX + NXL105 | 0.75 | 0.5* | 0.275* | 0.31* | 0.53 | 0.515 |
| P. aeruginosa QBR10 | ampC | CAZ + NXL105 | 0.531 | 0.281* | 0.156* | 0.091* | 0.061* | 0.0775* |
| | | MRP + NXL105 | 0.5075 | 0.265* | 0.156* | 0.091* | 0.093* | 0.0775* |
| | | LVX + NXL105 | 0.515 | 0.281* | 0.1875* | 0.073* | 0.043* | 1.015 |
| P. aeruginosa 391KB135 | ampC + IMP | CAZ + NXL105 | 0.75 | 0.5* | 0.375* | 0.31* | 0.53 | 0.515 |
| | | MRP + NXL105 | 0.75 | 0.5* | 0.375* | 0.56 | 0.53 | 0.515 |
| | | LVX + NXL105 | 0.625 | 0.38* | 0.375* | 0.56 | 0.53 | 0.515 |
| P. aeruginosa 391KB141 | ampC + VIM | CAZ + NXL105 | 0.75 | 0.5* | 0.625 | 1.06 | 1.03 | 1.015 |
| | | MRP + NXL105 | 0.75 | 0.5* | 0.375* | 0.31* | 0.53 | 0.515 |
| | | LVX + NXL105 | 0.625 | 0.75 | 0.625 | 0.56 | 0.53 | 1.015 |
| P. aeruginosa 391KB144 | ampC | CAZ + NXL105 | 0.625 | 0.5* | 0.375* | 0.31* | 0.253* | 0.515 |
| | | MRP + NXL105 | 0.625 | 0.5* | 0.625 | 0.56 | 0.53 | 0.515 |
| | | LVX + NXL105 | 0.625 | 0.75 | 0.625 | 0.56 | 0.53 | 1.015 |

FIC ≦ 0.5 = synergy, indicated with asterisk (*)

What is claimed is:

1. A combination with synergistic effect of a antibacterial compound of general formula (I) comprising: 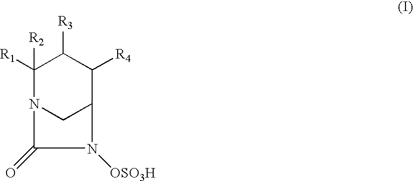
wherein R₁ represents a (CH₂)ₙ—NH₂ or (CH₂)ₙ—NHR radical, where R is a (C₁-C₆) alkyl and n is equal to 1 or 2;
R₂ represents a hydrogen atom;
R₃ and R₄ together form an aromatic nitrogenated heterocycle with 5 apexes with 1, 2 or 3 nitrogen atoms optionally substituted by one or several R′ groups, R′ being selected in the group composed of a hydrogen atom and the alkyl radicals with 1 to 6 carbon atoms;
in free form, as zwitterions, and in the form of salts of pharmaceutically acceptable inorganic or organic bases and acids;
with another antibacterial compound.

2. A combination according to claim 1, wherein the other antibacterial compound is selected among the group comprised of aminoglycosides, beta-lactams, monobactams, penicillins, if needed combined with a beta lactamases inhibitor, glycylcyclines, tetracyclines, quinolones, glycopeptides, lipopeptides, macrolides, ketolides, lincosamides, streptogramins, oxazolidinones, polymyxins and other compounds known to have therapeutic activity on Pseudomonas aeruginosa and Enterobacteriaceae.

3. A combination according to claim 1, wherein the compound of general formula (I), R₃ and R₄ together form a pyrazolyl or triazolyl heterocycle, optionally substituted.

4. A combination according to claim 1, wherein the compound of general formula (I), R₁ is selected in the group composed of the groups (CH₂)ₙ—NH₂ and (CH₂)ₙ—NHCH₃, where n is as defined in claim 1, the heterocycle formed by R₃ and R₄ is substituted by a (C₁-C₆) alkyl radical.

5. A combination according to claim 1, wherein the compound of general formula (I), R₁ represents a (CH₂)ₙ—NH₂ or (CH₂)ₙ—NHCH₃ radical, where n is as defined as in claim 1 and R₃ and R₄ together form a pyrazolyl ring substituted by a (C₁-C₆) alkyl radical.

6. A combination according to claim 1, wherein the compound of general formula (I) is one of the following:
trans 8-(aminomethyl)-4,8-dihydro-1-methyl-5-(sulphooxy)-4,7-methano-7H-pyrazolo[3,4-e][1,3]diazepin-6(5H)-one,
trans 8-(aminomethyl)-4,8-dihydro-5-(sulphooxy)-4,7-methano-7H-pyrazolo[3,4-e][1,3]diazepin-6(5H)-one,
trans 8-(methylaminomethyl)-4,8-dihydro-5-(sulphooxy)-4,7-methano-7H-pyrazolo[3,4-e][1,3]diazepin-6(5H)-one,
in its free form, as a zwitterion, and salts with pharmaceutically acceptable inorganic or organic bases and acids.

7. A combination according to claim 1, wherein the other antibacterial compound is selected among the group comprised of beta-lactams or penicillins, if needed combined with beta-lactamases inhibitors, aminoglycosides and polymyxins.

8. A combination according to claim 1, wherein the antibacterial compound is selected among the group comprised of tobramycin, meropenem, aztreonam, cefepime, ceftazidime, piperacillin, if needed combined with tazobactam, colistin and polymyxin B.

9. A combination according to claim 1, wherein the compound of general formula (I) is one of the following:
trans 8-(aminomethyl)-4,8-dihydro-1-methyl-5-(sulphooxy)-4,7-methano-7H-pyrazolo[3,4-e][1,3]diazepin-6(5H)-one,
trans 8-(aminomethyl)-4,8-dihydro-5-(sulphooxy)-4,7-methano-7H-pyrazolo[3,4-e][1,3]diazepin-6(5H)-one,
trans 8-(methylaminomethyl)-4,8-dihydro-5-(sulphooxy)-4,7-methano-7H-pyrazolo[3,4-e][1,3]diazepin-6(5H)-one,
in its free form, as a zwitterion, and salts with pharmaceutically acceptable inorganic or organic bases and acids;
and the antibacterial compound is selected among the group comprised of tobramycin, meropenem, cefepime, ceftazidime, aztreonam, levofloxacin, piperacillin, if needed combined with tazobactam, colistin and polymyxin B.

10. As drugs, the combinations as defined in claim 1.

11. As drugs, the combinations as defined in claim 9.

12. Pharmaceutical compositions containing, as active principle, at least one drug according to claim 11.

13. Pharmaceutical compositions containing, as active principle, at least one drug according to claim 12.

14. A pharmaceutical composition of general formula (I) comprising:
a combination with synergistic effect of a antibacterial compound of general formula (I) comprising: 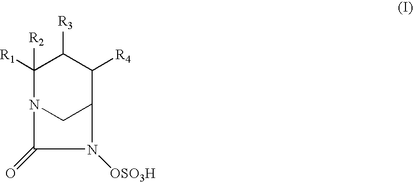
wherein R₁ represents a (CH₂)ₙ—NH₂ or (CH₂)ₙ—NHR radical, where R is a (C₁-C₆) alkyl and n is equal to 1 or 2;
R₂ represents a hydrogen atom;
R₃ and R₄ together form an aromatic nitrogenated heterocycle with 5 apexes with 1, 2 or 3 nitrogen atoms optionally substituted by one or several R′ groups, R′ being selected in the group composed of a hydrogen atom and the alkyl radicals with 1 to 6 carbon atoms;
in free form, as zwitterions, and in the form of salts of pharmaceutically acceptable inorganic or organic bases and acids; and
at least one of: (a) Psuedomonas aeruginosa; and (b) Enterobacteriaceae;
the composition having pharmaceutically antibacterial properties.
